(19) United States
(12) Patent Application Publication
(10) Pub. No.: US 2015/0133390 A1
(43) Pub. Date: May 14, 2015

(54) Identification of New Therapeutic Uses for Known Therapeutic Agents

(71) Applicant: The Board of Trustees of the Leland Stanford Junior University, Palo Alto, CA (US)

(72) Inventors: Purvesh Khatri, Stanford, CA (US); Atul J. Butte, Menlo Park, CA (US); Minnie M. Sarwal, Portola Valley, CA (US)

(73) Assignee: National Institutes of Health (NIH), U.S. Dept. of Health and Human Services (DHHS), U.S. Govt., Bethesda, MD (US)

(21) Appl. No.: 14/361,668

(22) PCT Filed: Jan. 25, 2013
(86) PCT No.: PCT/US2013/023292

§ 371 Date: May 29, 2014

Related U.S. Application Data
(60) Provisional application No. 61/591,403, filed on Jan. 27, 2012.

(51) Int. Cl. G01N 33/50; G06F 19/20; A61K 31/40; C12Q 1/68; A61K 31/506; A61K 31/4025

(52) U.S. Cl. CPC G01N 33/5023; A61K 31/506; A61K 31/4025; A61K 31/40; C12Q 1/6883; G06F 19/20; C12Q 2600/158; C12Q 2600/136; G01N 2500/10; G01N 2800/245
USPC 514/20.5; 514/252.19; 514/422; 514/427; 435/7.24; 506/9; 435/6.12; 435/6.13; 435/7.92; 436/501

(57) ABSTRACT

Methods for identifying new therapeutic activities for known therapeutic agents, as well as systems for practicing the same, are provided. Aspects of the invention further include are methods and compositions for the treatment of an acute graft rejection (AR).

### CROSS REFERENCE TO RELATED APPLICATIONS

Pursuant to 35 U.S.C. §119 (e), this application claims priority to the filing date of the U.S. Provisional Patent Application Ser. No. 61/591,403 filed Jan. 27, 2012; the disclosure of which is herein incorporated by reference.

### GOVERNMENT RIGHTS

This invention was made with government support under contracts LM009719, A1077821, and DK083447 awarded by the National Institutes of Health. The government has certain rights in this invention.

### INTRODUCTION

The development of a new drug to treat a condition is estimated to cost on average $1.3 billion USD. The estimated cost includes expenses for pre-clinical research, clinical trials, and obtaining regulatory approval to market the drug. In addition to great expenses associated with drug development, the process is a timely procedure. For example, a new cancer drug takes, on average, six years of research prior to reaching clinical trials. Moreover, on average, it takes another eight years from the time a cancer drug enters clinical trials until it receives approval from regulatory agencies for sale to the public. Drugs for other diseases have similar timelines.

### SUMMARY

Methods for identifying new therapeutic activities for known therapeutic agents, as well as systems for practicing the same, are provided. Aspects of the invention further include are methods and compositions for the treatment of an acute graft rejection (AR).

### BRIEF DESCRIPTION OF THE FIGURES

FIG. 1: depicts a meta-analysis workflow and experimental design of the experiment described in the Example below.

FIG. 2 shows an IPA regulatory network using 96 of the 102 genes, where each link in the network is supported by an experimental validation. Transcription factors STAT1 and NF-kappaB form a central axis of regulation of this network. Nodes highlighted in blue represent the 12 genes identified as common response module in solid organ rejection by leave-one-organ-out meta-analysis.

FIG. 3 depicts the Top 20 significant pathways using Ingenuity Pathway Analysis (IPA) for the 180 significant genes identified by meta-analysis in the Example below.

FIG. 4 depicts a chart showing that mapping of the 102 genes on the KEGG pathways, which were identified as significant by Pathway-Express, showed that these genes accurately recaptured the existing knowledge of the immune response during acute rejection. The genes are involved in various pathways that initiate immune response (antigen processing and presentation), which activate cell adhesion molecules as well as cytokine and cytokine receptors, which in turn activate a number of downstream signaling pathways (T-cell receptor signaling, Natural killer cell cytotoxicity) that ultimately lead to allograft rejection.

FIG. 5: shows a common rejection module consisting of 12 genes by leave-one-organ-out analysis. (A) Twelve genes were significantly overexpressed during acute rejection in all transplanted organs analyzed in this study, though they may not be significantly overexpressed in individual data sets. The x-axis represents standardized mean difference between AR and STA, computed as Hedges' g, in log₂ scale. The size of the blue rectangle represents the number of samples in the study. Whiskers represent the 95% confidence interval. The diamond represents overall, combined mean difference for a given gene. (B) Network analysis using MetaCore showed that 10 out of the 12 genes are part of a single regulatory network with NF-kappaB and STAT1 forming the central axis of regulation. Each interaction, except STAT1-ISG20, STAT1-BASP1, and GATA-3-NKG7, has been experimentally validated in the literature.

FIG. 6 shows a validation of the CRM genes in renal allografts. (A) All genes except CD7 were significantly overexpressed (p<0.005, FDR<2%) in two independent cohorts consisting of 383 renal transplant biopsies. (B-C) Distribution of CRM scores, defined as geometric mean of the CRIM genes expression, in AR and STA groups and ROC curve for GSE21374. (D-E) Distribution of CRM scores in AR and STA groups and ROC curve for the Stanford cohort. (F) CRM scores were significantly different between healthy control (HC), STA and AR. (G) CRM score increased with increase in graft injury.

FIG. 7 shows that overexpression of the 102 genes and the CRM gene set was validated in hearts from FVB mice transplanted in C57BL/6 WT mice. (A) 75 out of the 102 genes were significantly overexpressed (FDR<2%). (B) Entire CRM gene set was significantly overexpressed in the murine model (FDR<0.1%). (C-N) Expression of each of the CRM genes using Q-PCR in mice. *−p<0.05; **−p<0.005; ***−p<0.001.

FIG. 8 shows that atorvastatin and dasatinib treatment significantly extended allograft survival compared to untreated AR, both in mice and in humans. Each treatment group used 6 pairs of mice, where heart from FVB mouse was transplanted to C57BL/6 mouse. In total, we used 48 mice (24 pairs of FVB-to-057BL6 cardiac transplant). (A-E) Immunohistochemistry at POD 7 showed that the number of infiltrating cells in the cyclosporine, atorvastatin and dasatinib treatment groups was significantly reduced compared to untreated AR. (F-M) Number of infiltrating cells in cardiac allografts (in millions) in each group. Both atorvastatin and dasatinib significantly reduced the number of CD45+ infiltrating cells compared to the untreated AR group, and as much as cyclosporine. Atorvastatin and dasatinib also significantly reduced the number of infiltrating B220+ B cells and other antigen presenting cells, including F4/80+ macrophages, CD11c+ dendritic cells and NK1.1+ natural killer cells compared to untreated AR. *− statistically significant (p<0.05) reduction in the number of infiltrating cells compared to untreated AR group; +− statistically significant (p<0.05) reduction in the number of infiltrating cells compared to the cyclosporine group.

FIG. 9 shows that atorvastatin and dasatinib treatment significantly extended allograft survival compared to untreated AR, both in mice and in humans. Each treatment group used 6 pairs of mice, where heart from FVB mouse was transplanted to C57BL/6 mouse. In total, we used 48 mice (24 pairs of FVB-to-057BL6 cardiac transplant) (A) Median survival for untreated AR in mice was 10 days, whereas for atorvastatin, dasatinib, and cyclosporine it was 17 days (p=0.002), 24.5 days (p=0.0007), and 30 days (p=0.0002) respectively.

FIG. 10 depicts a retrospective analysis using electronic medical records of renal transplant patients showed that treating with statin increased the graft survival. Statin treatment started before or at 180 days post-transplantation was significantly associated with graft survival after the first 180 days in a cohort of 2,515 renal allograft recipients, censored for stopping statin therapy, graft failure and recipient death.

### TERMS

As used herein, the term “gene” or “recombinant gene” refers to a nucleic acid comprising an open reading frame encoding a polypeptide, including exon and (optionally) intron sequences. The term “intron” refers to a DNA sequence present in a given gene that is not translated into protein and is generally found between exons in a DNA molecule. In addition, a gene may optionally include its natural promoter (i.e., the promoter with which the exons and introns of the gene are operably linked in a non-recombinant cell, i.e., a naturally occurring cell), and associated regulatory sequences, and may or may not have sequences upstream of the AUG start site, and may or may not include untranslated leader sequences, signal sequences, downstream untranslated sequences, transcriptional start and stop sequences, polyadenylation signals, translational start and stop sequences, ribosome binding sites, and the like.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, procaryotic or eukaryotic mRNA, genomic DNA sequences from viral, procaryotic or eukaryotic DNA, and even synthetic DNA sequences. A transcription termination sequence may be located 3′ to the coding sequence.

The term “nucleic acid” includes DNA, RNA (double-stranded or single stranded), analogs (e.g., PNA or LNA molecules) and derivatives thereof. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “mRNA” means messenger RNA. An “oligonucleotide” generally refers to a nucleotide multimer of about 10 to 100 nucleotides in length, while a “polynucleotide” includes a nucleotide multimer having any number of nucleotides.

The terms “protein”, “polypeptide”, “peptide” and the like refer to a polymer of amino acids (an amino acid sequence) and does not refer to a specific length of the molecule. This term also refers to or includes any modifications of the polypeptide (e.g., post-translational), such as glycosylations, acetylations, phosphorylations and the like. Included within the definition are, for example, polypeptides containing one or more analogs of an amino acid, polypeptides with substituted linkages, as well as other modifications known in the art, both naturally occurring and non-naturally occurring.

The term “assessing” and “evaluating” are used interchangeably to refer to any form of measurement, and includes determining if an element is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery.

“Chronic transplant rejection or CR” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “transplant rejection” encompasses both acute and chronic transplant rejection.

### DETAILED DESCRIPTION

Methods for identifying new therapeutic activities for known therapeutic agents, as well as systems for practicing the same, are provided. Aspects of the invention further include are methods and compositions for the treatment of an acute graft rejection (AR).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods of Identifying New Therapeutic Activities for Known Therapeutic Agents

As summarized above, aspects of the invention include methods of identifying new therapeutic activities for known therapeutic agents, e.g., methods of determining novel therapeutic uses for known pharmaceutical compositions. In certain embodiments the method includes the steps of assessing samples from a plurality of subjects having a common condition for the presence of one or more biomarkers that are differentially expressed in the samples as compared to a control sample (e.g., by analyzing biomarker expression data from a plurality of subjects having a common condition for a biomarker that is differentially expressed in the subjects as compared to a control profile); identifying a known therapeutic agent that modulates the activity of at least one differentially expressed biomarker whose presence is determined by the assessing, wherein the known therapeutic agent is not known to have therapeutic activity for the common condition; and evaluating the therapeutic activity of the known therapeutic agent to treat the common condition. In some embodiments, the method further includes the steps of obtaining biomarker expression data from a plurality of subjects having a common condition.

As used herein “biomarker expression data” refers to data relating to the expression of one or more gene or protein biomarkers. In certain embodiments, the biomarker expression data includes data relating to the expression of one or more gene biomarkers. In other embodiments, biomarker expression data includes data relating to the expression of one or more protein biomarkers. In other embodiments, biomarker expression data includes data relating to the expression of one or more gene biomarkers and one or more protein biomarkers. In certain embodiments, the biomarker expression data includes data relating to the expression of one or more gene or protein biomarker obtained from two, three, four, five, six, seven, eight, nine, ten, eleven, twelve, thirteen, fourteen, fifteen, sixteen, seventeen, eighteen, nineteen, twenty, thirty, forty, fifty, sixty, seventy, eighty, ninety or one hundred or more studies.

In certain embodiments, the method includes the step of analyzing biomarker expression data from a plurality of subjects having a common condition for a biomarker that is differentially expressed in the plurality of subjects as compared to a control profile. As used herein, a “control profile” includes data relating to the expression of one or more biomarkers from a control subject that does not have the common condition. As such, analysis of the biomarker expression data for a biomarker that is differentially expressed in the plurality of subjects as compared to a control profile can lead to the identification of biomarkers that correlate with the common condition. In certain embodiments, the control profile includes data from two or more subjects. In certain embodiments, the control profile includes data from two or more studies.

Analysis of the biomarker expression data for a biomarker that is differentially expressed in the subjects as compared to a control profile can be performed using any suitable method, including those described in U.S. Pat. Nos. 6,308,170 and 6,228,575, the disclosures of which are herein incorporated by reference.

In certain embodiments, the analysis step is carried out using one or more meta-analysis methods. “Meta-analysis” refers to a method focused on contrasting and combining results from different studies, with a goal of identifying patterns among study results and/or sources of disagreement among those results. Models of meta-analysis include, but are not limited to, a fixed effect model, a random effects model, a quality effects model, a combining effect size model, and a combining p-value model.

In certain embodiments, the step of analyzing biomarker expression data is performed using a combining effect size model of meta-analysis. This combining effect size model allows for the estimation of the amount of change in biomarker expression across all studies in a biomarker expression dataset. In the combining effect size model, the effect size for each biomarker in the biomarker expression data and control profile is estimated as Hedges adjusted g (Hedges, Statistical methods for data analysis, Academic Press, (1985)). The study-specific effect sizes for each biomarker is then combined into a single meta effect-size using a linear combination of study-specific effect sizes, fᵢ, where each study-specific effect size is weighted by inverse of the variance in the corresponding study (Eq. 1). After computing meta effect-size, significant genes are identified using Z-statistic, and p-values were corrected for multiple hypotheses testing using Benjamini-Hochberg false discovery rate (FDR) correction (Benjamini and Hochberg, Journal of The Royal Statistical Society B 57: 289-300 (1995)).

$${f}_{meta} = \frac{{f}_{1} {w}_{1} + {f}_{2} {w}_{2} + … + {f}_{k} {w}_{k}}{{w}_{1} + {w}_{2} + … + {w}_{k}} ; {w}_{i} = \frac{1}{var ( {f}_{i} )} . \qquad Equation 1$$

In other embodiments, the step of analyzing biomarker expression data is performed using a combining p-value model of meta-analysis. This meta-analysis model allows for the determination of the statistical significance of a change in biomarker expression in each study in a biomarker expression dataset. In this model, p-values from each individual biomarker in the biomarker expression data is combined to identify biomarkers that have significantly large effect size in the biomarker expression data. For each biomarker, the logarithm of one-sided hypothesis testing p-values are summed across k studies and a comparison was performed of the results to a χ² distribution with 2k degrees of freedom (Eq. 2).

$${χ}_{2 k}^{2} = - 2 {\sum }_{i = 1}^{k} log ( {p}_{i )} . \qquad Equation 2$$

In certain embodiments, the step of analyzing biomarker expression data is performed using two or more meta-analysis models. In certain embodiments, the step of analyzing biomarker expression data is performed using a combining effect size model and a combining p-value model. In certain embodiments, the step of analyzing biomarker expression data is performed using two, three, four, five, six, seven, eight, nine, ten or more meta-analysis models. In some embodiments, a biomarker is deemed to be “differentially expressed” if a difference in expression level of the biomarker in the subjects having a common condition as compared to the control profile is observed in two or more of the meta-analysis models used in the analyzing step.

In certain embodiments, a biomarker expression data is compared to a single control profile to determine the expression level of a biomarker. In other embodiments, the biomarker expression data is compared to two or more different control profiles to obtain additional or more in depth information regarding the expression level of a biomarker. For example, the biomarker expression data may be compared to a positive and negative reference profile to obtain confirmed information regarding whether a particular biomarker is differentially expressed.

In some embodiments, the subject method further includes the steps of obtaining biomarker expression data from a subject having a condition prior to the step of analyzing the biomarker expression data. In certain embodiments, biomarker expression data is obtained by determining the expression level of a biomarker from a subject having a condition. In certain embodiments, biomarker expression data is obtained by determining the expression level of two, three, four, five, six, seven, eight, nine, ten or more biomarkers from a subject having a condition. In specific embodiments, the biomarker expression data is obtained from two or more subjects (i.e. a plurality of subjects) that have the condition (i.e. a common condition). The biomarker can be a gene or protein. The biomarker expression data can be quantitative or qualitative in nature. Any suitable gene and/or protein evaluation/quantitation protocol may be employed to obtain marker expression data including, but are not limited to: MRM analysis, standard immunoassays (e.g., ELISA assays, Western blots, FACS based protein analysis, etc.), protein activity assays, including multiplex protein activity assays, QPCR, expression arrays, etc.

Samples that are analyzed may vary, and include but are not limited to: tissue, blood, urine, and saliva samples. As detailed in the Experimental section below, tissue, blood or urine gene expression or urine protein analysis identified biomarkers that correlate with a particular condition. In certain embodiments, the sample is a tissue sample. In other embodiments, the sample is a blood sample. In other embodiments, the sample is a urine sample. In yet other embodiments, the sample is a saliva sample.

Where assessment of samples, e.g., as described above, results in identification of (i.e., determination of the presence or existence of) one or more commonly differentially expressed biomarkers, the methods may include a step of identifying a known pharmaceutical composition that targets (i.e., modulates the activity of) at least one of the identified differentially expressed biomarkers. In other words, the methods may include identifying a known therapeutic agent that modulates the activity of at least one differentially expressed biomarker whose presence is determined by the assessing. A known therapeutic agent (i.e., a known pharmaceutical composition) is an active agent that is not known to have therapeutic activity for the common condition of interest. The known therapeutic agent is one that is known to have therapeutic activity for a condition that is not the common condition of interest. In some instances, the known therapeutic agent is one that has been approved by a governmental agency, e.g., the United States Food and Drug Administration (FDA), for use in treatment of the condition that is not the target common condition. In certain embodiments, the known pharmaceutical composition has been clinically approved by a governmental or health agency or has gone through one or more phase or trials for clinical approval for a therapeutic use unrelated to the common condition. Such known drugs that have been clinically approved or gone through one or more phase or trials for clinical approval can advantageously allow for the repositioning of the known drugs for new conditions while minimizing the drug development costs associated with developing a new drug.

Identification of a known pharmaceutical composition that targets the differentially expressed biomarker can be performed using any suitable method. In certain embodiments, the identification step is carried out by performing a search using an internet search engine such as general web search engine (e.g., http://www.google.com) or a scientific literature search engine (e.g., http://www.ncbi.hlm.nih.gov).

In certain embodiments, the subject method includes the step of evaluating the therapeutic activity of the identified known therapeutic agent to treat the common condition (i.e., the ability of the known pharmaceutical composition to treat the common condition). Any suitable method may be used to assess therapeutic activity of the active agent for the common condition of interest may be employed, where such methods may include in vitro and/or in vivo methods. In certain embodiments, the evaluation step includes administering the known pharmaceutical composition to an animal model of the common condition and assessing whether symptoms of the common condition is reduced or eliminated in the animal model.

The common condition for which new therapeutic activity of known therapeutic agents may be assessed using methods as described herein may vary. In some instances, the common condition is a graft rejection condition, e.g., a allograft rejection condition, wherein the rejection may, in some instances, be an acute rejection, e.g., as described in greater detail below.

### Methods and Compositions for Treating Acute Graft Rejection

In some aspects, provided herein are methods for the treatment of an allograft rejection in a subject, wherein the method includes the step of administering to the subject a therapeutically effective amount of a common allograft response factor inhibitor or pharmaceutical composition thereof as described herein.

The terms “treatment”, “treating” and the like are used herein to generally mean obtaining a desired pharmacologic and/or physiologic effect. The effect may be prophylactic in terms of completely or partially preventing a disease or condition (e.g., an acute graft rejection) or symptom thereof and/or may be therapeutic in terms of a partial or complete cure for a disease or condition (e.g., an acute graft rejection) and/or adverse effect attributable to the disease. “Treatment” as used herein covers any treatment of a disease in a mammal, and includes: (a) preventing the disease or condition from occurring in a subject which may be predisposed to the disease but has not yet been diagnosed as having it; (b) inhibiting the disease or condition, i.e., arresting its development; or (c) relieving the disease, i.e., causing regression of the disease or condition. The common allograft response factor inhibitors may be administered before, during or after the onset of the disease or condition. The treatment of ongoing disease, where the treatment stabilizes or reduces the undesirable clinical symptoms of the patient, is of particular interest. Such treatment is desirably performed prior to complete loss of function in the affected tissues.

As used herein, a “common graft response factor” refers to any gene that is differentially expressed in subjects having an allograft rejection following an organ or tissue transplantation as compared to a control, regardless of the transplanted tissue or organ or polypeptide expressed from the gene.

In certain embodiments, the method described herein can be used to treat any type of allograft rejection associated with the transplantation of any organ or tissue. In certain embodiments, the organ is selected from kidney, heart, liver, lung, intestine, pancreas, eye, skin, bone marrow, and other organs. In certain embodiments, the organ is selected from kidney, heart, liver, and lung. In certain embodiments, the organ is kidney. In certain embodiments, the organ is heart. In certain embodiments, the organ is liver. In certain embodiments, the organ is lung.

In specific embodiments, the method of treatment is for the treatment of an acute graft rejection. By an “acute allograft rejection”, an “acute graft rejection” or “AR” is meant a graft rejection that occurs over a period of 7-10 days (in a primary response) or 2-3 days (in a secondary response). Acute graft rejection involves rejection involves both cell-mediated and antibody-mediated immunity. In such embodiments, the subject is administered an effective amount of a common acute graft response factor inhibitor. As used herein, a “common acute graft response factor”, “common rejection module” and “CRM” all refer to any gene/protein that is differentially expressed in subjects having an acute graft rejection following an organ or tissue transplantation as compared to a control, regardless of the transplanted tissue or organ. As disclosed in the Experimental section below, common acute graft response factors include, but are not limited to, BASP1, CD6, CD7, CXCL10, CXCL9, INPP5D, ISG20, LCK, NKG7, PSMB9, RUNX3 and TAP1.

The subject methods may be employed with a variety of different types of transplant subjects. In many embodiments, the subjects are within the class mammalian, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the animals or hosts, i.e., subjects (also referred to herein as patients) are humans.

As disclosed herein, inhibitors of common acute graft response factors include, but are not limited to, tyrosine kinase inhibitors (e.g., BCR/ABL and Src family tyrosine kinase inhibitors), statins (i.e., HMG-CoA reductase inhibitors), and antibodies that selectively bind to a common acute graft response factor (e.g., anti-CXCL10).

In certain embodiments of the method, the common acute graft response factor inhibitor is a tyrosine kinase inhibitor. In certain embodiments, the tyrosine kinase inhibitor is a Src family tyrosine kinase inhibitor. In specific embodiments, the Src tyrosine kinase inhibitor is an amido substituted thiazole amine. In certain embodiments, the Src tyrosine kinase inhibitor has the formula:

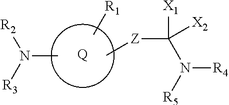

wherein

Q is thiazole;

Z is a single bond;

X₁ and X₂ together form ═O;

R₁ is hydrogen or alkyl;

R₂ is hydrogen or alkly;

R₃ is —Z₄—Z₆ wherein Z₄ is a single bond and wherein Z₆ is heteroaryl substituted with at least one group Z₃,

R₄ is hydrogen or alkly; and

R₅ is aryl which is unsubstitute or substitute with Z₁, Z₂ and one or more groups Z₃, and

Z₁, Z₂ and Z₃ are each independently
(1) hydrogen or Z₅, where Z₅ is (i) alkyl, alkenyl, alkynyl, cycoalkyl, cycloalkylalkyl, cycloalkenyl, cycloalkenylalkyl, aryl, aralkyl, alkylaryl, cycloalkylaryl, heterocyclo, or heterocycloalkyl; (ii) a group (i) which is itself substituted by one or more of the same or different groups (i); or (iii) a group (i) or (ii) which is substituted by one or more of the following groups (2) to (16) of the definition of Z₁, Z₂ and Z₃;
(2) —OH or —OZ₅;
(3) —SH or —SZ₅;
(4) —SH or —SZ₅;
(5) halo;
(6) cyano;
(7) nitro;
(8) oxo
(9) —O—C(O)—Z₅;
(10) any two of Z₁, Z₂ and Z₃ may together be alkylene or alkenylene completing a 3- to 8-membered saturated or unsaturated ring together with the atoms to which they are attached; or
(11) any two of Z₁, Z₂ and Z₃ may together be —O—(CH₂)ᵣ—O—, where r is 1 to 5, completing a 4- to 8-membered ring together with the atoms to which they are attached.

In specific embodiments, the Src family tyrosine kinase inhibitor is dasatinib. Dasatinib is a drug for treatment of non-transplant conditions. Dasatinib (BMS-354825, Sprycel; Bristol-Meyers Squibb, New York, N.Y., USA) is an ATP-competitor that has been approved for the treatment of Imatinib-resistant chronic myeloid leukemia. Dasatinib has the following structure:

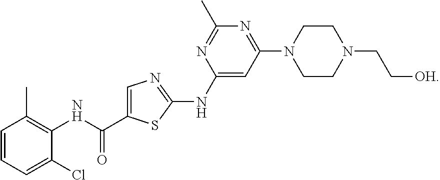 [SMILES: Cc1nc(Nc2ncc(C(=O)Nc3c(C)cccc3Cl)s2)cc(N2CCN(CCO)CC2)n1]

In other embodiments of the methods, the common acute graft response factor inhibitor is a statin. Statins (or HMG-COA reductase inhibitors) are a class of drugs used to lower cholesterol levels by inhibiting the enzyme HMG-CoA reductase. Statins include, but are not limited to, atorvastatin, fluvastatin, lovastatin, pitavastatin, pravastatin, rosuvastatin, and simvastatin. In certain embodiments, the statin is a trans-6-[2-(3- or 4-carboxamido-substitute pyrrol-1-yl)alkyl]-4-hydroxypyran-2one or a ring-opened acid derivative thereof. In certain embodiments, the statin has the formula:

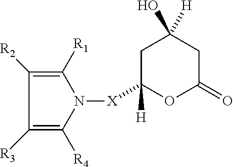

wherein

X is —CH₂—, —CH₂CH₂—, —CH₂CH₂CH₂—, or —CH₂CH(CH₃)—

R₁ is 1-naphtyl; 2-napthyl; cyclohexyl; norbornenyl; phenyl; phenyl substituted with fluorine; chlorine; bromine; hydroxyl; trifluoromethyl; alkyl of from one to four carbon atoms; alkoxy of from one to four carbon atoms; or alkanoyloxy of from two to eight carbon atoms;

either of R₂ or R₃ is —CONR₅R₆ where R₅ and R₆ are independently hydrogen; alkyl of form one to six carbon atoms; phenyl; phenyl substituted with fluorine, chlorine, bromine, cyano, trifluoromethyl, or carboalkoxy of from three to eight carbon atoms;

and the other of R₂ or R₃ is hydrogen; of from one to six carbon atoms; cyclopropyl; cyclobutyl; cyclopentyl; cyclohexyl; phenyl; or phenyl substituted with fluorine, chlorine, bromine, hydroxyl, trifluoromethyl, alkyl of from one to four carbon atoms, alkoxy of from one to four carbon atoms, or alkanoyloxy of from two to eight carbon atoms;

R₄ is alkyl of from one to six carbon atoms; cyclopropyl; cyclobutyl; cyclopentyl; cyclohexyl; or trifluoromethyl; or a hydroxyl acid or pharmaceutically acceptable salts thereof, corresponding to the opened ring of the compounds having the formula.

In specific embodiments, the statin is atorvastatin. Atorvastatin is a drug for treatment of non-transplant conditions. Atorvastatin is an HMG-CoA reductase inhibitor that slows the production of cholesterol. Atorvastatin has the following structure:

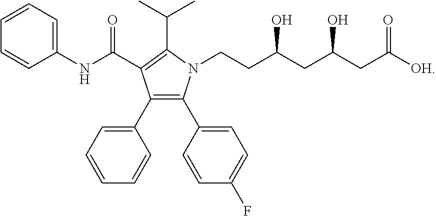 [SMILES: CC(C)c1c(C(=O)Nc2ccccc2)c(-c2ccccc2)c(-c2ccc(F)cc2)n1CC[C@@H](O)C[C@@H](O)CC(=O)O]

In certain embodiments, the common acute graft response factor inhibitor is a specific binding member, such as an antibody or binding fragment thereof. Specific binding members of interest include, but are not limited to, specific binding members that bind to a common acute graft response factor selected, such as a factor selected from the group consisting of: BASP1, CD6, CD7, CXCL10, CXCL9, INPP5D, ISG20, LCK, NKG7, PSMB9, RUNX3 and TAP1. In certain embodiments, the method comprises administering to the subject a therapeutically effective amount of a specific binding member that targets, i.e., modulates the activity of, PSMB9, CXCR3, CXCL10, or INPP5D. In some instances, the antibody targets CXCL10. In some instances, the antibody is MDX-1100. MDX-1100 (Medarex, Princeton, N.J.) is a fully human monoclonal antibody that binds selectively to CXCL10.

For inclusion in a medicament, the common acute graft response factor inhibitor or pharmaceutical composition thereof may be obtained from a suitable commercial source. For example, atorvastatin is commercially available as LIPITOR (Pfizer, New York, N.Y., USA). Dasatinib is commercially available as SPRYCEL (Bristol-Myers Squibb, New York, N.Y., USA). Anti-CXCL10 antibody is commercially available as MDX-1100 (Medarex, Princeton, N.J., USA).

By a “therapeutically effective amount” of a common acute graft response factor inhibitor is meant an amount that is required to reduce the severity, the duration and/or the symptoms of a disease or condition (e.g., acute graft rejection). Symptoms of an acute graft rejection include, but are not limited to, necrosis of parenchymal cells in a graft associated with lymphocyte and macrophage infiltrates; macrophage-mediated cell lysis, natural killer (NK) cell mediated lysis, necrosis of parenchymal cells or endothelial cells in the graft; and vasculitis. In certain embodiments, a therapeutically effect amount is an amount that is required to reduce the number of immune cell (e.g., CD4+ T cells, CD8+ T cells, B cells, macrophages, dendritic cells and natural killer cells) that infiltrate a graft.

The effective amount of a common acute graft response factor inhibitor or pharmaceutical composition thereof to be given to a particular patient will depend on a variety of factors, several of which will differ from patient to patient. For example, the effective amount may be dependent upon the route of administration, the seriousness of the graft rejection, and should be decided according to the judgment of the practitioner and each human patient's circumstances.

Determining a therapeutically effective amount of the common acute graft response factor inhibitor can be done based on animal data using routine computational methods. For example, effective amounts may be extrapolated from dose-response curves derived from preclinical protocols either in vitro or using any suitable in vivo allograft rejection animal models (e.g., a heart, kidney, liver, or lung transplantation animal model, see, e.g., Bumgardner et al., Transplatation 68(4): 555-562 (1999), De Vleeschauwer et al., Transplant. Proc. 43(9): 3476-3485 (2011); Ge et al., Exp Clin Transplant 9(5): 287-94 (2011); and Schwenger et al., Nephroi. Dial. Transplant 22(suppl 8): viii47-viii49 (2007), incorporated herein by reference). Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration.

In some embodiments, the therapeutically effective amount of the pharmaceutical composition provided herein is between about 0.025 mg/kg and about 1000 mg/kg body weight of a human subject. In certain embodiments, the pharmaceutical composition is administered to a human subject at an amount of about 1000 mg/kg body weight or less, about 950 mg/kg body weight or less, about 900 mg/kg body weight or less, about 850 mg/kg body weight or less, about 800 mg/kg body weight or less, about 750 mg/kg body weight or less, about 700 mg/kg body weight or less, about 650 mg/kg body weight or less, about 600 mg/kg body weight or less, about 550 mg/kg body weight or less, about 500 mg/kg body weight or less, about 450 mg/kg body weight or less, about 400 mg/kg body weight or less, about 350 mg/kg body weight or less, about 300 mg/kg body weight or less, about 250 mg/kg body weight or less, about 200 mg/kg body weight or less, about 150 mg/kg body weight or less, about 100 mg/kg body weight or less, about 95 mg/kg body weight or less, about 90 mg/kg body weight or less, about 85 mg/kg body weight or less, about 80 mg/kg body weight or less, about 75 mg/kg body weight or less, about 70 mg/kg body weight or less, or about 65 mg/kg body weight or less.

In some embodiments, the effective amount of the pharmaceutical composition provided herein is between about 0.025 mg/kg and about 60 mg/kg body weight of a human subject. In some embodiments, the effective amount of an antibody of the pharmaceutical composition provided herein is about 0.025 mg/kg or less, about 0.05 mg/kg or less, about 0.10 mg/kg or less, about 0.20 mg/kg or less, about 0.40 mg/kg or less, about 0.80 mg/kg or less, about 1.0 mg/kg or less, about 1.5 mg/kg or less, about 3 mg/kg or less, about 5 mg/kg or less, about 1 0 mg/kg or less, about 15 mg/kg or less, about 20 mg/kg or less, about 25 mg/kg or less, about 30 mg/kg or less, about 35 mg/kg or less, about 40 mg/kg or less, about 45 mg/kg or less, about 50 mg/kg or about 60 mg/kg or less.

In some embodiments, the method further includes the step of evaluating the subject after administering the pharmaceutical composition to determine whether an effective amount of an inhibitor has been administered. In certain embodiments, the evaluation step includes determining graft infiltration by immune cells. Such a determination can be made by histological analysis of a graft sample obtained from a biopsy. See, e.g., Martinu et al., Proc Am Thorac Soc 15(6): 54-65 (2009); Josephson, Clin J Am Soc Nephrol 6(7): 1774-80 (2011); and Mengel et al., Am J Transplant 10(9): 2105-15 (2010). In other embodiments, the evaluation step includes determining graft function after administration of the pharmaceutical composition. For example, lung function can be determined using a pulmonary function test such as spirometry testing. See, e.g., Keenan et al., J Thorac Cardiovasc Surg 113: 335-341 (1997). Liver function tests can include measurements of albumin, alpha-1 antitrypsin, alkaline phosphatase (ALP), alanine transaminase (ALT), gamma-glutamyl transpeptide (GGT), prothrombin time, and/or serum or urine bilirubin. See e.g., Pincus and Abraham, Henry's Clinical Diagnosis and Management by Laboratory Methods, Chapter 8, 21st ed. Philadelphia, Pa.: Saunders Elsevier (2006). Kidney function can be assessed, for example, by monitoring serum creatine levels serially. See e.g., Josephson, Clin J Am Soc Nephrol 6(7): 1774-80 (2011). Heart function can be assessed by any suitable method, including performing an electrocardiogram or echocardiogram on the subject. Jurt et al., Circulation 106: 1750-1752 (2002).

The common acute graft response factor inhibitors and pharmaceutical compositions thereof can be administered for prophylactic and/or therapeutic treatments. In certain embodiments, the common acute graft response factor inhibitor is administered prior to graft transplantation. In other embodiments, the common acute graft response factor inhibitor is administered concurrently with graft transplantation. In yet other embodiments, the common acute graft response factor inhibitor is administered after graft transplantation.

Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized. The inhibitor or pharmaceutical compositions thereof be administered daily, semi-weekly, weekly, semi-monthly, monthly, etc., at a dose of from about 0.01 mg, from about 0.1 mg, from about 1 mg, from about 5 mg, from about 10 mg, from about 100 mg or more per kilogram of body weight when administered systemically. Smaller doses may be utilized in localized administration, e.g., in direct administration to ocular nerves, etc.

In some embodiments, a graft is contacted in vivo with one or more of the common acute graft response factor inhibitors. Cells in vivo may be contacted with one or inhibitors suitable for pharmaceutical use, by any of a number of well-known methods in the art for the administration of polypeptides and nucleic acids to a subject. The common acute graft response factor inhibitor can be incorporated into a variety of formulations. More particularly, the common acute graft response factor inhibitor can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the common acute graft response factor inhibitor or pharmaceutical composition thereof can be achieved in various ways, including oral, buccal, rectal, parenteral, intraperitoneal, intradermal, transdermal, intracheal, etc., administration. The pharmaceutical composition comprising the inhibitor may be systemic after administration or may be localized by the use of regional administration, intramural administration, or use of an implant that acts to retain the active dose at the site of implantation. The pharmaceutical composition comprising the common acute graft response factor inhibitor may be formulated for immediate activity or they may be formulated for sustained release.

A common acute graft response factor inhibitor for pharmaceutical use, i.e. a common acute graft response factor inhibitor pharmaceutical composition, can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The pharmaceutical composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The common acute graft response factor inhibitor or pharmaceutical composition thereof to be used for therapeutic administration must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The common acute graft response factor inhibitor or pharmaceutical composition thereof ordinarily will be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The pharmaceutical composition comprising the lyophilized common acute graft response factor inhibitor(s) is prepared by reconstituting the lyophilized compound, for example, by using bacteriostatic Water-for-Injection.

Where desired, an active agent as described above may be administered in combination with a second active agent that exhibits therapeutic activity for the target condition. By “in combination with” is meant that an amount of a first active agent is administered together with an amount of a second active agent that is different from the first active agent, e.g., has a different molecular formula from the active agent. In certain embodiments, the first and second active agents are administered sequentially. In yet other embodiments, the first and second active agents are administered simultaneously, e.g., where the first and second agents are administered at the same time as two separate formulations or are combined into a single composition that is administered to the subject. Regardless of whether the first and second active agents are administered sequentially or simultaneously, as illustrated above, the agents are considered to be administered together or in combination for purposes of the present invention. Routes of administration of the two agents may vary, where representative routes of administration are described in greater detail below. In certain embodiments, the method includes administering to the subject two or more common acute graft response factor inhibitors or a pharmaceutical composition that includes two or more common acute graft response factor inhibitors. In some embodiments, two, three, four, five, six, seven, eight, nine or ten common acute graft response factor inhibitors are administered to the subject. In some instances, the common acute graft response factor inhibitor(s) is administered in combination with known immunosuppressive agent. Known immunosuppressive agents include, but are not limited, rapamycin, cyclosporin A, anti-CD40L monoclonal antibody, and the like.

Mammalian species that may be treated with the present methods include canines and felines; equines; bovines; ovines; etc. and primates, particularly humans. In some embodiments, the method is for the treatment of a human. Animal models, particularly small mammals, e.g., murine, lagomorpha, etc. may be used for experimental investigations. Of interest are subjects or patients that have are going to receive or have received an allograft transplant, such that the subject is a subject that is known to be in need of allograft rejection therapy.

### PHARMACEUTICAL COMPOSITIONS

In some aspects, provided herein are pharmaceutical compositions for the treatment of an allograft rejection wherein the pharmaceutical composition includes a therapeutically effective amount of a common acute graft response factor inhibitor (e.g., dasatinib, atorvastatin, an anti-CXCL10 antibody) provided herein, together with a suitable amount of carrier so as to provide the form for proper administration to a subject. In particular embodiments, the pharmaceutical composition is for the treatment of an acute graft rejection.

In certain embodiments, the pharmaceutical composition described herein can be used to treat any type of allograft rejection associated with the transplantation of any organ or tissue, e.g., a solid organ graft. In certain embodiments, the organ is selected from kidney, heart, liver, lung, intestine, pancreas, eye, skin, bone marrow, and other organs. In certain embodiments, the organ is selected from kidney, heart, liver, and lung. In certain embodiments, the organ is kidney. In certain embodiments, the organ is heart. In certain embodiments, the organ is liver. In certain embodiments, the organ is lung.

By a “therapeutically effective amount” of a common acute graft response factor inhibitor it is meant an amount that is required to reduce the severity, the duration and/or the symptoms of a disease or condition (e.g., acute graft rejection). Symptoms of an acute graft rejection include, but are not limited to, necrosis of parenchymal cells in a graft associated with lymphocyte and macrophage infiltrates; macrophage-mediated cell lysis, natural killer (NK) cell mediated lysis, necrosis of parenchymal cells or endothelial cells in the graft; and vasculitis. In certain embodiments, a therapeutically effect amount is an amount that is required to reduce the number of immune cell (e.g., CD4+ T cells, CD8+ T cells, B cells, macrophages, dendritic cells and natural killer cells) that infiltrate a graft. A therapeutically effective amount can be determined by any suitable method including, but not limited to, the methods described above.

In some embodiments, the therapeutically effective amount of the pharmaceutical composition is between about 0.025 mg/kg and about 1000 mg/kg body weight of a human subject. In certain embodiments, the pharmaceutical composition is administered to a human subject at an amount of about 1000 mg/kg body weight or less, about 950 mg/kg body weight or less, about 900 mg/kg body weight or less, about 850 mg/kg body weight or less, about 800 mg/kg body weight or less, about 750 mg/kg body weight or less, about 700 mg/kg body weight or less, about 650 mg/kg body weight or less, about 600 mg/kg body weight or less, about 550 mg/kg body weight or less, about 500 mg/kg body weight or less, about 450 mg/kg body weight or less, about 400 mg/kg body weight or less, about 350 mg/kg body weight or less, about 300 mg/kg body weight or less, about 250 mg/kg body weight or less, about 200 mg/kg body weight or less, about 150 mg/kg body weight or less, about 100 mg/kg body weight or less, about 95 mg/kg body weight or less, about 90 mg/kg body weight or less, about 85 mg/kg body weight or less, about 80 mg/kg body weight or less, about 75 mg/kg body weight or less, about 70 mg/kg body weight or less, or about 65 mg/kg body weight or less.

In some embodiments, the therapeutically effective amount of the pharmaceutical composition provided herein is between about 0.025 mg/kg and about 60 mg/kg body weight of a human subject. In some embodiments, the effective amount of an antibody of the pharmaceutical composition provided herein is about 0.025 mg/kg or less, about 0.05 mg/kg or less, about 0.10 mg/kg or less, about 0.20 mg/kg or less, about 0.40 mg/kg or less, about 0.80 mg/kg or less, about 1.0 mg/kg or less, about 1.5 mg/kg or less, about 3 mg/kg or less, about 5 mg/kg or less, about 1 0 mg/kg or less, about 15 mg/kg or less, about 20 mg/kg or less, about 25 mg/kg or less, about 30 mg/kg or less, about 35 mg/kg or less, about 40 mg/kg or less, about 45 mg/kg or less, about 50 mg/kg or about 60 mg/kg or less.

Inhibitors of common acute graft response factors that may be used in the pharmaceutical compositions provided herein include, but are not limited to, tyrosine kinase inhibitors (e.g., BCR/ABL and Src family tyrosine kinase inhibitors), statins (i.e., HMG-CoA reductase inhibitors), and antibodies that selectively bind to a common acute graft response factor (e.g., anti-CXCL10), e.g., as described above.

The term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the PUM1 or SF2 inhibitor is administered. Such pharmaceutical carriers can be, for example, sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The inhibitors can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin, hereby incorporated by reference herein in its entirety. Such compositions will contain a therapeutically effective amount of the Pumilio-like protein (e.g., PUM1) or SR protein (e.g., SF2) inhibitor, preferably in purified form, together with a suitable amount of carrier so as to provide the form for proper administration to the patient. The formulation should suit the mode of administration.

The pharmaceutical composition can be formulated for intravenous, oral, via implant, transmucosal, transdermal, intramuscular, intrathecal, or subcutaneous administration. In some embodiments, the pharmaceutical composition is formulated for intravenous administration. In other embodiments, the pharmaceutical composition is formulated for subcutaneous administration. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGAs). Implantable systems include rods and discs, and can contain excipients such as PLGA and polycaprylactone. Osteopontin or nucleic acids of the invention can also be administered attached to particles using a gene gun.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, the pharmaceutical composition is supplied as a water free concentrate. In some embodiments, the pharmaceutical composition is supplied as a dry sterile lyophilized powder at a unit dosage of at least 0.5 mg, at least 1 mg, at least 2 mg, at least 3 mg, at least 5 mg, at least 1 0 mg, at least 15 mg, at least 25 mg, at least 30 mg, at least 35 mg, at least 45 mg, at least 50 mg, at least 60 mg, or at least 75 mg.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. In certain embodiments, the pharmaceutical composition includes two or more common acute graft response factor inhibitors. In some embodiments, two, three, four, five, six, seven, eight, nine or ten common acute graft response factor inhibitors are administered to the subject. In some instances, the pharmaceutical composition further includes a known immunosuppressive agent. Known immunosuppressive agents include, but are not limited, rapamycin, cyclosporin A, anti-CD40L monoclonal antibody, and the like.

### Utility

The subject methods for determining novel therapeutic uses for a known pharmaceutical composition provided herein find use in a variety of different applications where development of new therapies for a particular condition is desired. In particular embodiments provided herein, the known pharmaceutical composition has been clinically approved by a governmental or health agency or has gone through one or more phase or trials for clinical approval for a therapeutic use unrelated to the particular condition. Thus, such methods can advantageously allow for the repositioning of the known drugs for new conditions while minimizing the drug development costs as well as the lengthy period of time associated with developing a new drug. As shown in the Examples provided herein, such methods have been used to determine a therapeutic use for the treatment of an acute allograft rejection in an organ transplant recipient for at least two known drugs—dasatinib and atorvastatin—that are FDA approved for non-transplant conditions.

Also provided herein are methods of treatments and composition for the treatment of an acute graft rejection that targets genes identified as involved in a common acute graft rejection mechanism. These new treatments allow when performed alone or administered with existing therapies contribute to an increased rate of graft transplantation. Further, such methods and compositions allow for diagnostics and therapeutics relating to organ or tissue graft rejection without requiring details about tissue-specific injuries. Moreover, the use of known drugs in these methods help to alleviate the escalating costs related to drug discovery associated with the smaller organ transplantation disease market.

### Computer Systems, Devices and Computer-Readable Media

Steps of the subject methods can be computer-implemented, such that method steps (e.g., assaying, comparing, calculating, identifying, and/or the like) are automated in whole or in part. Accordingly, the present disclosure provides computer systems, devices, computer readable media and the like configured to implement the methods or portions thereof.

For example, the methods of the present disclosure may involve inputting expression data into a computer programmed to execute an algorithm to identify one or more candidate pharmaceutical agents based thereon, and generate a report as described herein, e.g., by displaying or printing a report to an output device at a location local or remote to the computer.

The present disclosure thus provides a computer program product including a computer-readable storage medium having a computer program stored on it. The program can, when read by a computer, execute relevant calculations based on gene expression values (e.g., relating to the identity of the gene and level of expression thereof) and barcode information obtained from analysis of one or more target cells in the cellular sample. The computer program product has stored therein a computer program for performing the calculation(s).

Systems for executing the program described above are also provided. The systems may include: a) a central computing environment; b) an input device, operatively connected to the computing environment, to receive gene expression data, where the gene expression data can include, e.g., sequence data that includes gene-specific and barcode-specific sequence information, as well as data indicative of the abundance of a gene expression product or panel of gene expression products of interest, and/or any other useful values obtained from an assay using the target cell(s) within the cellular sample, as described above; and c) an output device, connected to the computing environment, to provide information to a user (e.g., medical or research personnel). In certain aspects, the system further includes an algorithm executed by the central computing environment (e.g., a processor), where the algorithm is executed based on the data received by the input device, and wherein the algorithm calculates a value, which value is indicative of the biological condition of the transduced target cell.

Computer systems may include a processing system, which may include at least one processor or processing unit or plurality of processors, memory, at least one input device and at least one output device, coupled together via a bus or group of buses. In certain embodiments, an input device and output device can be the same device. The memory can be any form of memory device, for example, volatile or non-volatile memory, solid state storage devices, magnetic devices, etc. The processor can comprise more than one distinct processing device, for example to handle different functions within the processing system.

An input device receives input data and can comprise, for example, a keyboard, a pointer device such as a pen-like device or a mouse, audio receiving device for voice controlled activation such as a microphone, data receiver or antenna such as a modem or wireless data adaptor, data acquisition card, etc. Input data can come from different sources, for example keyboard instructions in conjunction with data received via a network.

Output devices produce or generate output data and can comprise, for example, a display device or monitor in which case output data is visual, a printer in which case output data is printed, a port for example a USB port, a peripheral component adaptor, a data transmitter or antenna such as a modem or wireless network adaptor, etc. Output data can be distinct and derived from different output devices, for example a visual display on a monitor in conjunction with data transmitted to a network. A user can view data output, or an interpretation of the data output, on, for example, a monitor or using a printer. The storage device can be any form of data or information storage means, for example, volatile or non-volatile memory, solid state storage devices, magnetic devices, etc.

In use, the processing system may be adapted to allow data or information to be stored in and/or retrieved from, via wired or wireless communication means, at least one database. The interface may allow wired and/or wireless communication between the processing unit and peripheral components that may serve a specialized purpose. In general, the processor can receive instructions as input data via input device and can display processed results or other output to a user by utilizing output device. More than one input device and/or output device can be provided. A processing system may be any suitable form of terminal, server, specialized hardware, or the like.

Computer programs (also known as programs, software, software applications, applications, components, or code) include instructions for a programmable processor, and may be implemented in a high-level procedural and/or object-oriented programming language, and/or in assembly/machine language. As used herein, the term “computer-readable medium” refers to any computer program product, apparatus and/or device (e.g., magnetic discs, optical disks, memory, etc.) used to provide machine instructions and/or data to a programmable processor, including a machine-readable medium that receives machine instructions as a machine-readable signal.

Aspects of the present disclosure may be embodied, at least in part, in software, hardware, firmware, or any combination thereof. Thus, the techniques described herein are not limited to any specific combination of hardware circuitry and/or software, or to any particular source for the instructions executed by a computer or other data processing system. Rather, these techniques may be carried out in a computer system or other data processing system in response to one or more processors, such as a microprocessor, executing sequences of instructions stored in memory or other computer-readable medium including any type of ROM, RAM, cache memory, network memory, floppy disks, hard drive disk (HDD), solid-state devices (SSD), optical disk, CD-ROM, and magnetic-optical disk, EPROMs, EEPROMs, flash memory, or any other type of media suitable for storing instructions in electronic format.

The following examples are offered by way of illustration and not by way of limitation.

### EXPERIMENTAL

### I. Introduction

Bioinformatics meta-analysis of eight transcriptional datasets comprised of 236 graft biopsy samples from four types of transplanted organs were performed. The meta-analysis led to the identification of a common rejection module (CRM) consisting of 11 genes that were significantly overexpressed (p<0.0005) during biopsy confirmed acute rejection, irrespective of the transplanted organ. Overexpression of the CRM genes were validated in three independent cohorts consisting of 503 human renal transplant biopsies. Using pathway analysis and inferred drug mechanisms from an extensive literature search, two FDA-approved drugs (atorvastatin and dasatinib), approved for non-transplant indications, were identified as potential regulators of specific CRM genes that can reduce the number of graft infiltrating cells during acute rejection. Confirmation of the ability of atorvastatin and dasatinib to modulate the CRM genes and significantly reduce graft infiltrating cells during acute rejection and extended graft survival was carried out using an HLA-mismatched murine cardiac transplant model treated with atorvastatin and dasatinib. Further validation of the beneficial effect of atorvastatin on graft survival by retrospective analysis of electronic medical records of a single-center cohort of 2,515 renal transplant patients followed for up to 22 years. From this study, a common rejection module in organ transplantation across organs and species was identified, which provides new opportunities for drug repositioning and rational drug design.

### II. Materials and Methods

### A. Data Collection and Pre-Processing

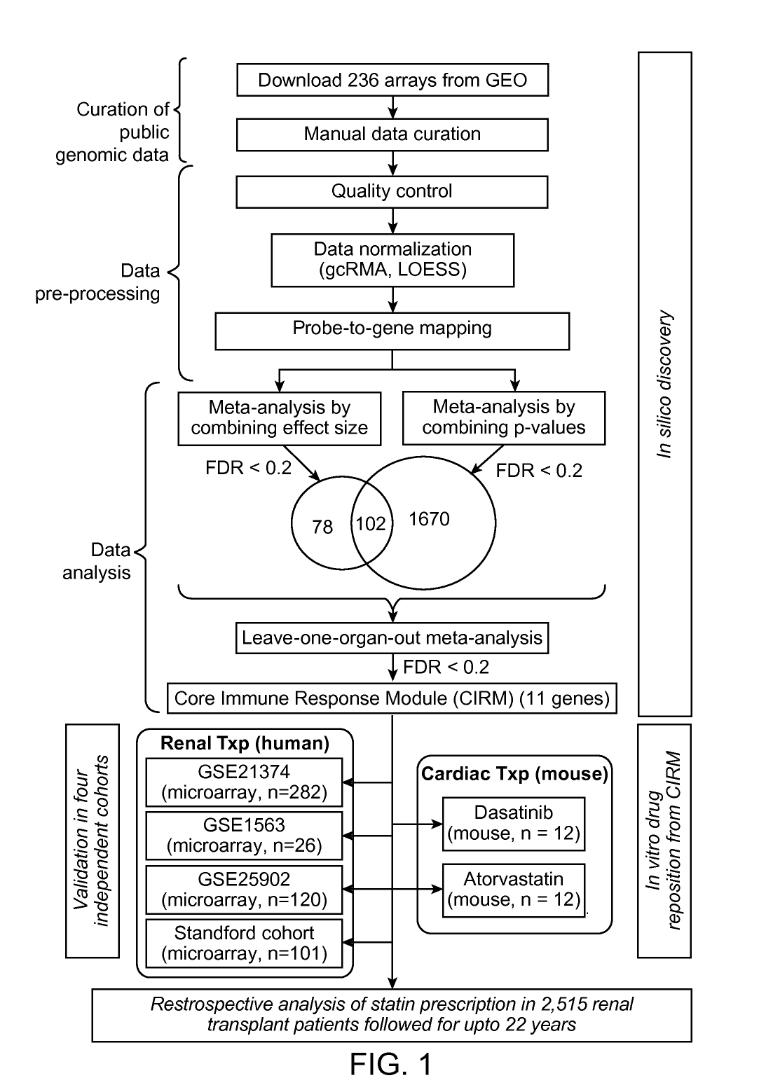

As shown in FIG. 1, we downloaded eight transplant gene expression data sets from four solid organs from GEO. Each data set was manually curated to select only the tissue biopsy samples from AR and STA patients. All oligonucleotide arrays were checked for quality to ensure that the arrays were free of any experimental artifacts. Microarrays from cDNA-based platform were not checked as raw image files were not available from GEO. Each oligonucleotide data set was normalized using gcRMA. Microarray probes in each data set were mapped to Entrez Gene identifiers (IDs) to facilitate meta-analysis. If a probe matched more than one gene, the expression data for the probe were expanded to add one record for each mapped gene.

B. Meta-Analysis by Combining Effect Size and p-Values

The eight solid organ transplant data sets were analyzed using two different meta-analysis methods: i) combining effect size and ii) combining p-values. We estimated the effect size for each gene in each data set as Hedges' g³, which is analogous to fold-change estimate. The study-specific effect sizes for each gene were then combined into a single meta effect-size using a linear combination of study-specific effect sizes, fᵢ, where each study-specific effect size was weighted by inverse of the variance in the corresponding study (Eq. 1). After computing meta effect-size, significant genes were identified using Z-statistic, and p-values were corrected for multiple hypotheses testing using Benjamini-Hochberg FDR correction.

$${f}_{meta} = \frac{{f}_{1} {w}_{1} + {f}_{2} {w}_{2} + … + {f}_{k} {w}_{k}}{{w}_{1} + {w}_{2} + … + {w}_{k}} ; {w}_{i} = \frac{1}{var ( {f}_{i} )}$$

We used Fisher's sum of logs method for meta-analysis by combining p-values. Using this method, for each gene, we summed the logarithm of the (one-sided hypothesis testing) p-values across k studies, and compared that to a χ²-distribution with 2k degrees of freedom to identify significant genes (Eq. 2).

$${χ}_{2 k}^{2} = - 2 {\sum }_{i = 1}^{k} log ( {p}_{i )}$$

### C. Selection of 102 Significant Genes

We selected 102 genes that satisfied following criteria: (1) meta effect size >0 (i.e., over-expressed genes), (2) when combining effect size, FDR<20% across all data sets, (3) measured in all 8 data sets, (4) when combining p-values using Fisher's test, p-value<0.2 for a gene to be up-regulated.

### D. Leave-One-Organ-Out Analysis

In order to account for the unequal number of data sets for each organ as well as to find the set of genes that are over-expressed in solid organ transplant independent of the source organ, we performed meta-analysis by removing all data sets corresponding to one organ at a time. For instance, in the first iteration, all data sets from heart (GDS1684, GSE2596, GSE4470, and GSE9377) were removed, and meta-analysis was performed using only the data sets from kidney, lung and liver. In the second iteration, all data sets from kidney were removed and meta-analysis was performed using the data sets from heart, lung and liver.

At each iteration, i.e., after removing data sets for one organ at a time, we performed meta-analysis by combining effect-sizes (Eq. 1) and by combining p-values (Eq. 2). Using FDR<20% as threshold, at each iteration we identified 12 genes that were expressed in all data sets used in the given iteration.

### E. Functional Pathway Analysis

We performed functional pathway analysis using Pathway-Express (PE). We used meta effect-size as fold change in PE to identify significant pathways. We used FDR<0.1 as a threshold for identifying significant pathways. We performed network analysis using IPA with option to only include “direct relationship” in order to avoid spurious connections due to “indirect relations”.

### F. QRT-PCR to Confirm CIRM Module Genes and Effect of Drug Treatment on CIRM Module Genes in Mouse

PBMC were isolated using Ficoll (Ficoll-Paque™ PLUS, Amersham Biosciences, Uppsala, Sweden) from 5 healthy individuals (2 female, 3 males, mean age 31+/−14 years) counted, and an average of 1.2×105 cells/well were plated on a 98 well plate (U-Bottom, Nunc, Roskilde, DK) in a total volume of 280 μL RPMI 1640 supplemented with 10% FCS, Pen/Strep (100 U/mL) and 2% non-essential Amino acids (all Gibco®, Invitrogen, Life Technologies, CA, USA). Anti-Biotin MACSi™ Bead Particles were bound to human CD3/− CD28-Biotin (1 μg antigen/108 beads) according to the manufactures protocol (MACS human T-cell activation/expansion kit, Miltenyi Biotec) for 2 hours at 4° C. on a rotator. Thereafter, 20 μL of CD3/CD28 bound beads (2.5×107 loaded beads/mL) in RPMI 1640 were added for T-cell stimulation. For the unstimulated cells an equal amount of the above medium only was added. After 68 hours cells were centrifuged, washed with ice cold PBS (Gibco®, Invitrogen, Life Technologies, CA, USA). Total RNA from cells were isolated using the Quiagen RNeasy mini Kit plus 50 (Quiagen Sciences, Maryland, USA) and reverse transcribed using Superscript II (Invitrogen, Life Technologies, CA, USA). For CIRM module gene expression analysis, the following TaqMan primers and probes (Applied Biosystems, Life Technologies, Foster City, Calif.) were used: LCK (Hs00894952_g1), PSMB9 (Hs00544762_m1), RUNX3 (Hs00231709_m1), ISG20 (Hs00158122_m1); INPP5D (Hs00183290_m1), CD6 (Hs00198752_m1); CD7 (Hs00196191_m1); NKG7 (Hs01120688_g1); BASP1 (Hs00932356_s1); TAP1 (Hs00388675_m1); CXCL9 (Hs00171065_m1); CXCL10 (Hs00171042_m1). Samples were analyzed in duplicates (12 ng RNA per well) for a total of 40 cycles. S18 (Ha03003631_g1) served as endogenous control, and universal human reference RNA (Stratagene, La Jolla, Calif., USA) was used for relative quantification of gene expression.

QRT-PCR for the CIRM module genes in mouse allografts was performed using a high-throughput RT-PCR instrument (BioMark; Fluidigm, San Francisco, Calif.). Total RNA was extracted from flash frozen apical graft portions (one third of the allograft) using TRIzol® Reagent (Invitrogen, Life Technologies, Carlsbad, Calif.) according to standard protocols. cDNA generated using Superscript II (Invitrogen, Carlsbad) was preamplified on an Eppenford Thermocycler for the CIRM modules genes. Preamplified cDNA was mixed with TaqMan® Universal PCR Master Mix (Applied Biosystems) and Sample Loading Reagent (http://www.fluidigm.com/, San Francisco, Calif.) and pipetted into the sample inlets of a Dynamic Array 96.96 chip (Fluidigm). TaqMan gene expression assays (Applied Biosystems) for the 12 genes plus 18S as endogenous control gene were diluted with Assay Loading Reagent (1:2) (Fluidigm) and pipetted into the assay inlets of the same Dynamic Array 96.96 chip. After distributing assays and samples into the reaction wells of the chip in the NanoFlex controller (Fluidigm), a total of 1,876 qRT-PCR reactions were performed in the BioMark RT PCR system in a total of 40 cycles. Data was analyzed using the BioMark RT-PCR Analysis Software Version 2.0. Using the delta Ct method, gene expression in each sample was calculated relative to the expression in a universal RNA sample (human universal RNA, Stratagene, CA). The IDs of the assays used in the PCR are as follows: 18S (Hs99999901_s1); BASP1 (Mm0234432_s1); CD6 (Mm01208285_m1); CD7 (Mm00438111_m1); CXCL9 (Mm00434946_m1); CXCL10 (Mm00445235_m1); INPP5D (Mm00494987_m1); ISG20 (Mm00469585_m1); LCK (Mm0080297_m1); NKG7 (Mm00452524_g1); PSMB9 (Mm00479004_m1); RUNX3 (Mm00490666_m1); and TAP1 (Mm00443188_m1).

### G. Microarray Profiling

i. Human Renal Allograft Biopsies

For each kidney allograft biopsy, a separate core was stored in RNAlater (Ambion, Austin, Tex.) and stored at −20° C. until RNA extraction. Total RNA was extracted from each biopsy using TRIzol Reagent (Invitrogen, Carlsbad, Calif.). RNA integrity was ensured using the RNA 6000 Nano LabChip Kit (Agilent Technologies, Waldbronn, Germany) on a 2100 Bioanalyzer (Agilent Technologies). RNA was amplified to cDNA and biotin labeled using the Ovation Biotin System (NuGEN Technologies, San Carlos, Calif.). The cDNA fragments were hybridized onto Affymetrix GeneChip Human Genome U133 Plus 2.0 Arrays comprising more than 54,000 probe sets, covering more than 47,000 transcripts and variants, including 38,500 well characterized human genes (Affymetrix, Santa Clara, Calif.). The microarrays were scanned using GeneChip Scanner 3000 (Affymetrix).

ii. Mouse Heart Allografts

For whole mouse genome expression analysis, Agilent Whole Mouse Genome 4×44K 60mer oligonucleotide arrays (G2519F, Agilent Technologies, Palo Alto, Calif.) were used. A total of 100 ng total RNA was used in the Agilent LIRAK PLUS, two-color Low RNA input Linear Amplification method, according to the manufacturer's instructions. Briefly, first the total RNA was reverse transcribed into complimentary DNA (cDNA) using T7-promotor primer and MMLV reverse transcriptase. The cDNA was transcribed into complimentary RNA (cRNA), during which it was fluorescently labeled by incorporation of cyanine (Cy)5-CTP (exposed samples) or Cy3-CTP (negative control samples). After purification, using the RNeasy mini kit (Qiagen), cRNA yield and Cy incorporation efficiency (specific activity) into the cRNA were determined using a NanoDrop Spectrophotometer (NanoDrop Technologies). cRNAs showing a yield >825 ng and a specific activity of 8-20 pmol/μg were selected for further processing. Equal amounts of the exposed and negative control sample (825 ng) were competitively hybridized onto Agilent Whole Mouse Gene expression microarray GE 4×44 koligonucleotide arrays in a hybridization oven at 65° C. for 17 hours. Slides were washed according to the manufacturer's instructions with washing buffers and finally dipped in Stabilization and Drying Solution (Agilent Technologies) to protect them from environmental ozone. The arrays were scanned on an Agilent scanner and further processed using Agilent Feature Extraction Software. Agilent universal mouse reference RNA (#740100, Agilent Technologies, Palo Alto, Calif.) was used as reference sample.

### H. Animals and Heterotopic Heart Transplantation

C57BL/6J (H2^{b}) and FVB (H2^{q}) mice were purchased from Jackson Laboratory (Bar Harbor, Me.). Male mice (6-10 wk) with an average body weight of 25 g were used in the experiments. Animals were maintained in the animal care facility at Stanford University, and all experiments were approved by the Stanford University Institutional Animal Care and performed in accordance with the Guide for the Care and Use of Laboratory Animals.

FVB donor hearts were implanted into the abdomen of C57BL/6 WT mice representing a complete MHC mismatch, as described previously. Animals were divided into three treatment groups (Atorvastatin, Dasatinib, and Cyclosporine) and one non-treated control group, each consisting of 6 animals. Animal activity, body weight and graft viability (abdominal palpation) were assessed daily.

### I. Drugs and Treatment

For mice treatment, commercially available Atorvastatin (PZ0001; Sigma Aldrich, St. Louis, Mo.) and Dasatinib (S1021; Selleck Chemicals LLC, Houston, Tex.) were suspended in sterile PBS (AccuGENE; Lonza Rockland Inc, Rockland, Me.) at concentrations of 9 mg/mL and 13.5 mg/mL for low and high dose Atorvastatin, respectively, and at a concentration of 4.5 mg/mL for Dasatinib. Drug suspensions were aliquoted and stored at 4° C. with respect to Atorvastatin and at −20° C. with respect to Dasatinib. For intraperitoneal application of Cyclosporine, Cyclosporine 250 mg/mL in ethanol and polyoxyethylated castor oil USP grade for i.v. injection (Bedford Labs™, Bedford, Ohio) was ordered through Stanford Hospital Pharmacy and diluted to 1 mg/mL in sterile saline solution.

Atorvastatin (75 mg/kg body weight/day) and Dasatinib (25 mg/kg body weight/day) were administered daily by oral gavages, Cyclosporine (20 mg/kg body weight/day) was administered daily intraperitoneally. Before oral gavages of Atorvastatin and Dasatinib, aliquots were mixed thoroughly. Unused formulations were discarded. Treatment started the day prior to transplantation and lasted until the day before sacrifice at post operation day 7 by exsanguination. Grafts were harvested and divided into three equal parts for downstream analyses.

For survival study, another group of 6 mice for each treatment (total 18 mice for Cyclosporine, Atorvastatin, and Dasatinib) and 6 mice for untreated AR group were used using the same protocol described above. Graft viability (abdominal palpation) were assessed daily for these animals until post operation day 30.

### J. Histology

One third of the explanted allograft heart (POD7) was immediately fixed in 20% buffered formalin, embedded in paraffin and subsequently stained with hematoxylin and eosin for histological assessment of tissue according to standard protocols. Pictures of the graft tissue were taken at 10× magnification using a Nikon E600 light microscope (Nikon Instruments Inc., Melville, N.Y.) and Spot V4.6 imaging software (Spot Imaging, Sterling Heights, Mich.).

### K. Flow Cytometry Analysis

Fluorescein isothiocyanate (FITC), phycoerythin (PE), or allophycocyanin-conjugated mAbs specific for mouse CD4 (GK1.5), CD8a (53-6.7), F4/80 (BM8), B220 (RA3-6B2), Gr1 (RB6-8C5), CD11c (N418), NK1.1 (PK136), CD45 (30-F11), and their isotype controls were purchased from BD Biosciences (San Jose, Calif.), eBioscience (San Diego, Calif.), or BioLegend (San Diego, Calif.). Immediately after graft explantation at day 7 post transplantation, one third of the cardiac allograft was homogenized in RPMI 1640 media with 2 mg/mL Collagenase D (Worthington Bio) and 10% FCS for 2 h at RT. Cells were first incubated with normal hamster serum, normal mouse serum (Jackson ImmunoResearch), and 5 μg/ml anti-CD16/32 mAb (2.4G2; BD Bioscience), then stained with FITC-, PE-, and APC-conjugated mAbs for 30 min at 4° C. To exclude the dead cells, 7-Amino-Actinomycin D (BD Bioscience) was added and incubated for 10 minutes before analysis. Expression of markers was determined by FACS Calibur (BD Bioscience) and FlowJo software (Tree Star).

### III. RESULTS

### A. Meta-Analysis of Solid Organ Transplants Data Sets Recapitulates Known Mechanisms of Acute Rejection.

Raw data was downloaded for eight gene expression studies from organ biopsy specimens from kidney, lung, heart and liver transplant patients, with and without diagnosis of AR (FIG. 1). Each data set was filtered to include only biopsy data from patients with AR and patients who were in stable condition. After re-annotating the probes, each data set was separately normalized using gcRMA (Irizarry et al., 2003, Nucleic Acids Res. 31:e15). Data sets GSE2596 and GSE4470 were not normalized because raw data were not available, and the downloaded data were already normalized.

Two meta-analyses approaches were applied to the normalized data. Briefly, the first approach combines effect sizes from each dataset into a meta-effect size to estimate the amount of change in expression across all data sets. For each gene in each data set, an effect size was computed using Hedges' adjusted g. If multiple probes mapped to a gene, the effect size for each gene was summarized using the fixed effect inverse-variance model. Study-specific effect sizes were combined to obtain the pooled effect size and its standard error using the random effects inverse-variance technique. Z-statistics were then computed as a ratio of the pooled effect size to its standard error for each gene, and compared the result to a standard normal distribution to obtain a nominal p-value. P-values were corrected for multiple hypotheses testing using FDR (Storey, 2002). We identified 180 genes that were measured in all data sets and were overexpressed in AR with p<0.01 (FDR≦20%).

A second non-parametric meta-analysis was used that combines p-values from individual experiments to identify genes with a large effect size in all data sets. Briefly, a t-statistic for each gene in each study was calculated. After computing one-tail p-values for each gene, they were corrected for multiple hypotheses using FDR. Next, Fisher's sum of logs method (Fisher, 1932), which sums the logarithm of corrected p-values across all data sets for each gene, was used and compares the sum against a chi-square distribution with 2k degrees of freedom, where k is the number of data sets used in the analysis. This method identified 1772 overexpressed genes at FDR<20%).

One hundred two genes were identified as significantly overexpressed by both methods (FIG. 1). This group contains genes that (i) had a large effect size in all data sets and (ii) were consistently significant across all data sets. Although the selection criteria used may have left out genes with varying expression in AR, the method allowed for the development of robust overlapping transcriptional signals in AR across all transplanted organs.

Using BioGPS, a genes was defined as preferentially expressed in a tissue, if its expression in a given tissue was at least three times its median expression across all 84 tissues in BioGPS (Su et al., 2004). It was determined that the 102 genes are highly expressed in one or more blood cell types that participate in the immune response, suggesting that the meta-analysis removed tissue-specific bias and identified the relevant pathogenic transcriptional signal of activated infiltrating cells in the graft in AR, rather than being affected by various confounding factors such as organ-specific expression bias, treatment protocols by different groups, or different microarray platforms.

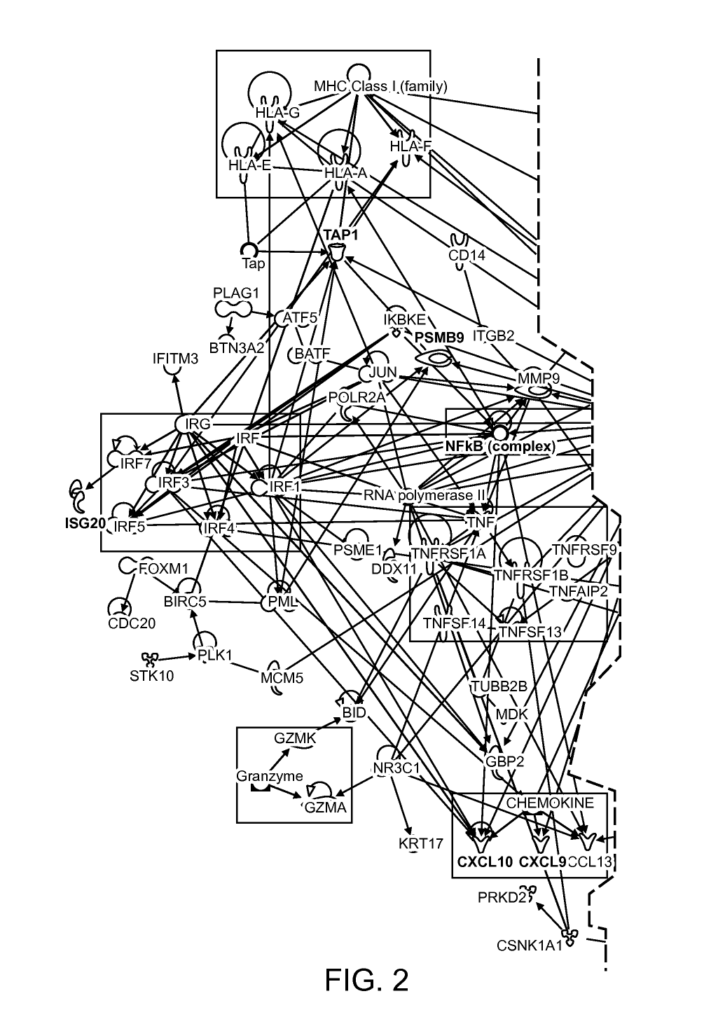

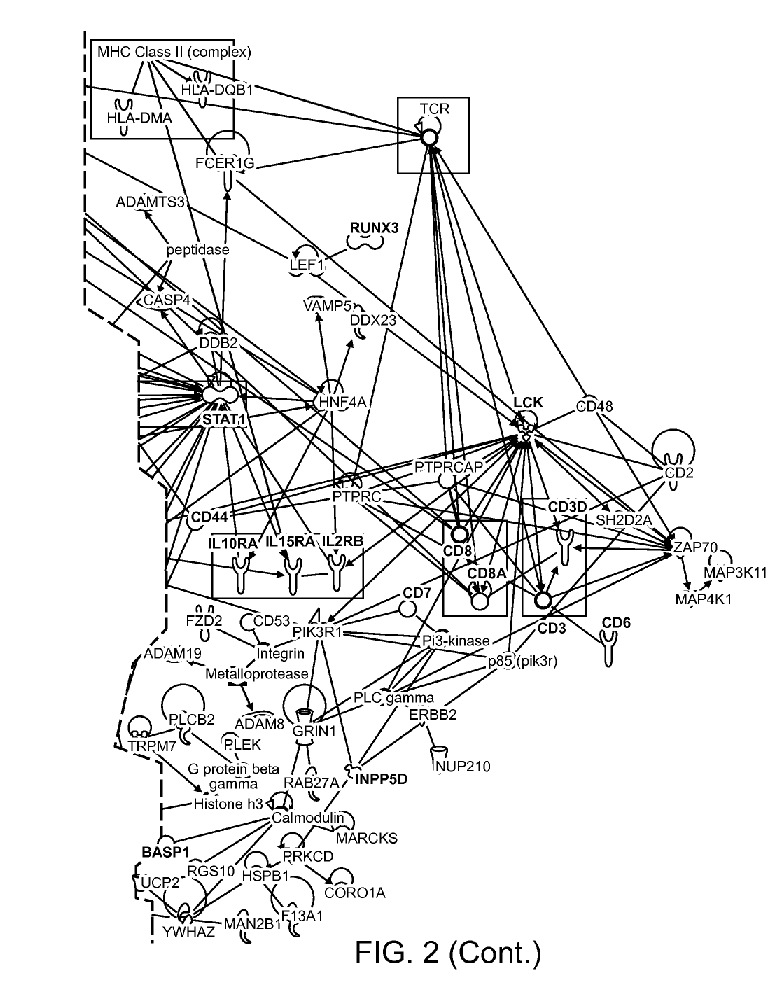

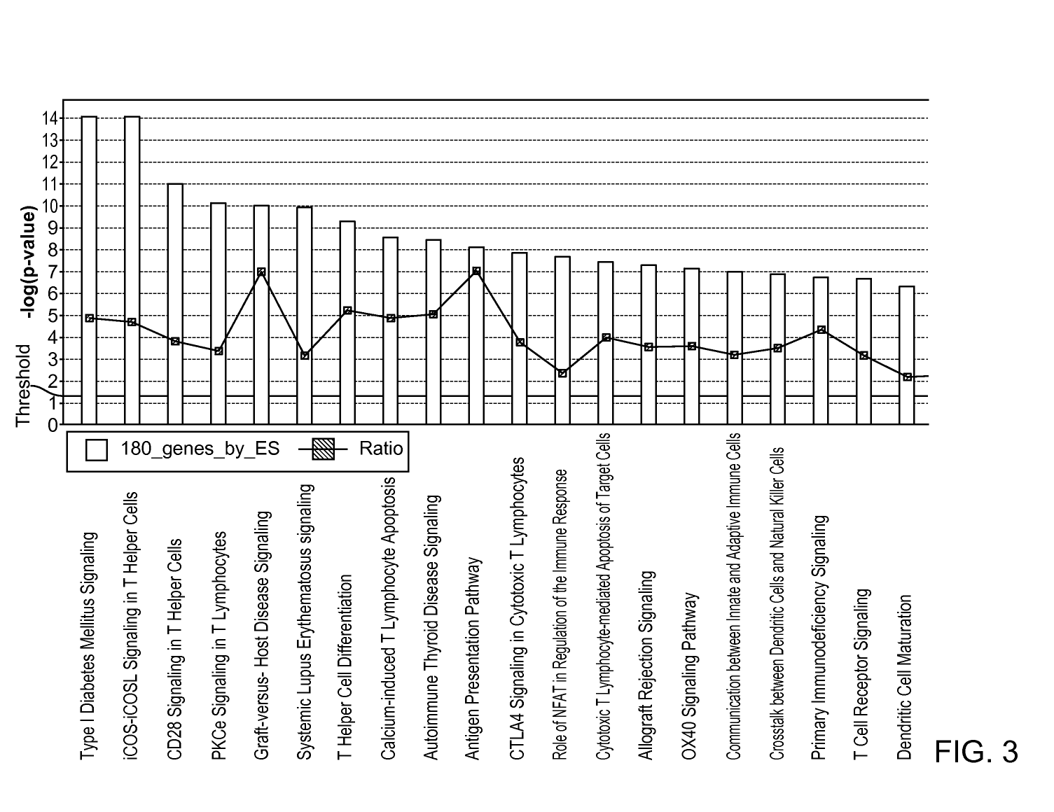

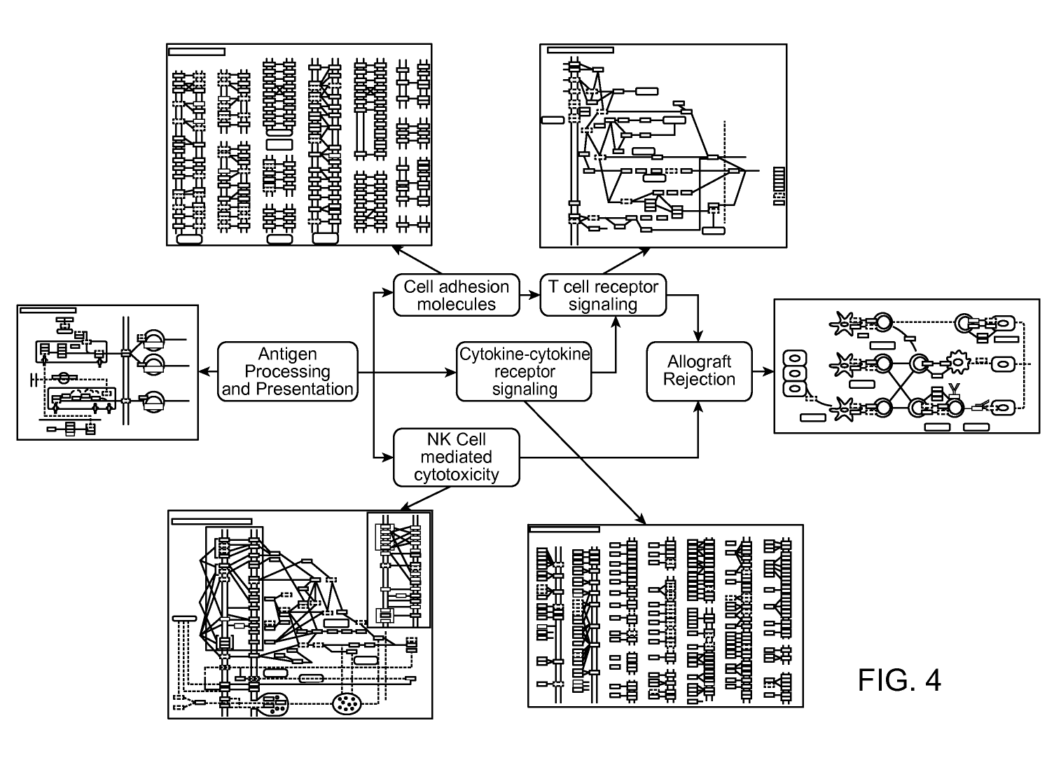

Network analysis of the 102 genes using Ingenuity Pathway Analysis (IPA) revealed that 96 of the genes are part of a network involved in cellular movement and immune cell trafficking. These genes include major histocompatibility complex class I and II molecules, interferon regulatory factors, granzymes, chemokines, interleukins, transcription factors and the T cell receptors. All have direct relationships to one another, and each relationship has been experimentally verified in the literature (FIG. 2). Canonical pathway analysis of these overexpressed genes using IPA and Pathway-Express (Draghici et al., 2007, Genome Research. 17:1537-1545; Khatri et al., 2008, In Progress in Pattern Recognition, Image Analysis and Applications. Springer Berlin) confirmed that they are in many of the pathways known to be related to regulation of the immune response and our current understanding of graft rejection (FIGS. 3 and 4).

### B. Meta-Analysis Using Leave-One-Organ-Out Identifies Ubiquitously Overexpressed Genes in Allograft Rejection.

In order to avoid (1) the influence of a single large experiment on the meta-analysis results and (2) organ-specific bias due to unequal number of data sets (and samples) used in the meta-analysis, a “leave-one-organ-out” (LOOO) meta-analysis was performed. All data sets from individual organs were excluded, one organ at a time, and meta-analysis was performed on the remaining data sets from three organs.

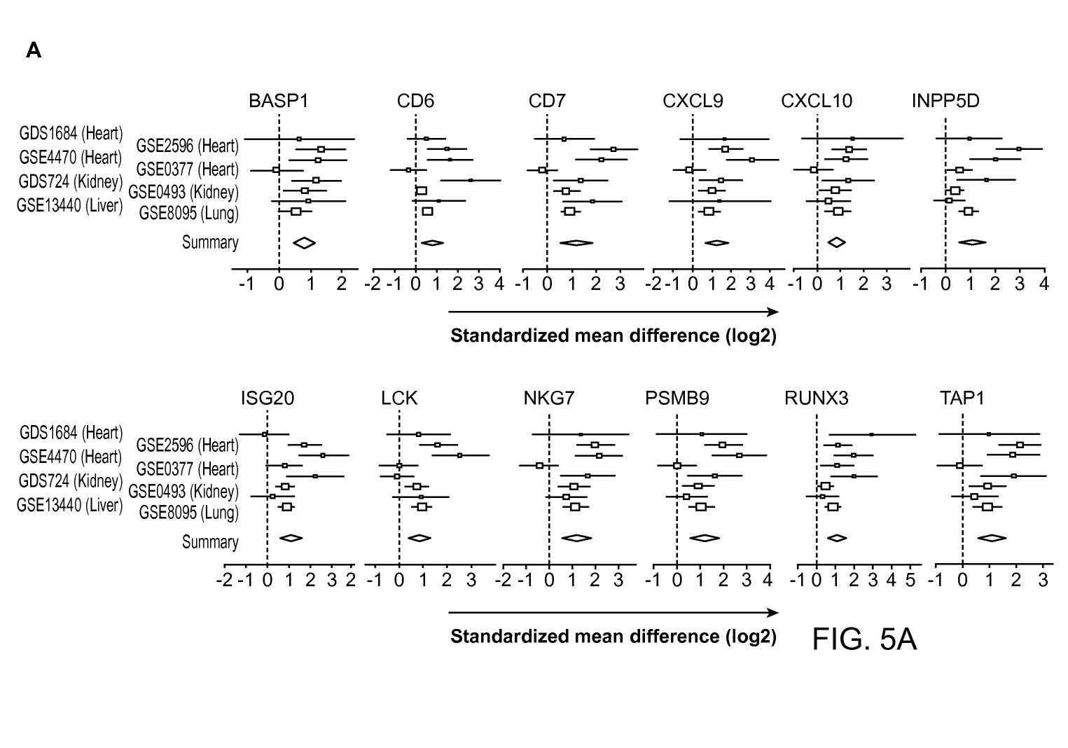

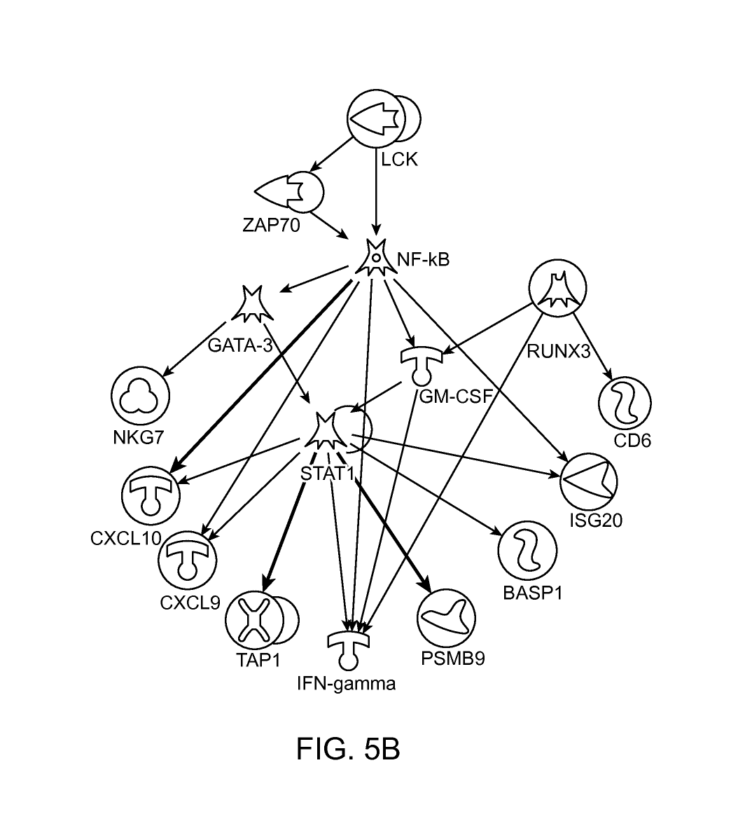

From the analysis, 12 overexpressed genes were identified: BASP1, CD6, CD7, CXCL10, CXCL9, INPP5D, ISG20, LCK, NKG7, PSMB9, RUNX3, and TAP1 at FDR≦20% (FIG. 5A). Network analysis using MetaCore (www.genego.com) showed that 10 out of the 12 genes are connected to each other, where STAT1 and NF-kB form central axis of regulation (FIG. 5B). Regulation of expression by STAT1 and NK-kB for many genes in this network has been verified experimentally in the literature (Sharif et al., 2004, Journal of immunology 172:6476-6481; Shi et al., 2005, Journal of Immunology 175:3318-3328; Ellis et al., 2010, Journal of immunology 185(3): 1864-77; Chatterjee-Kishore et al., 1998, J Biol Chem. 273:16177-16183; Der et al., 1998, Proc. Natl. Acad. of Sci. 26:15623-15628; Kuznetsov, 2009, Transplantation. 61:1469-1474; Robertson et al., 2007, Nature Methods. 4:651-657).

### C. Validation in Two Independent Cohorts of 383 Renal Transplant Patients.

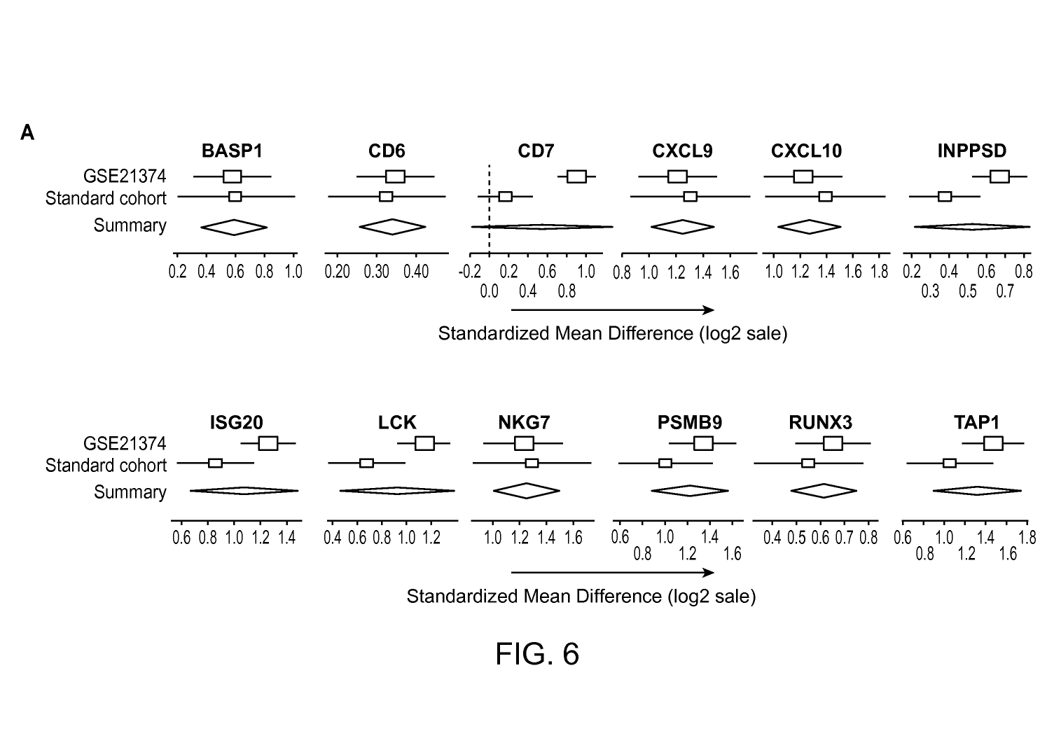

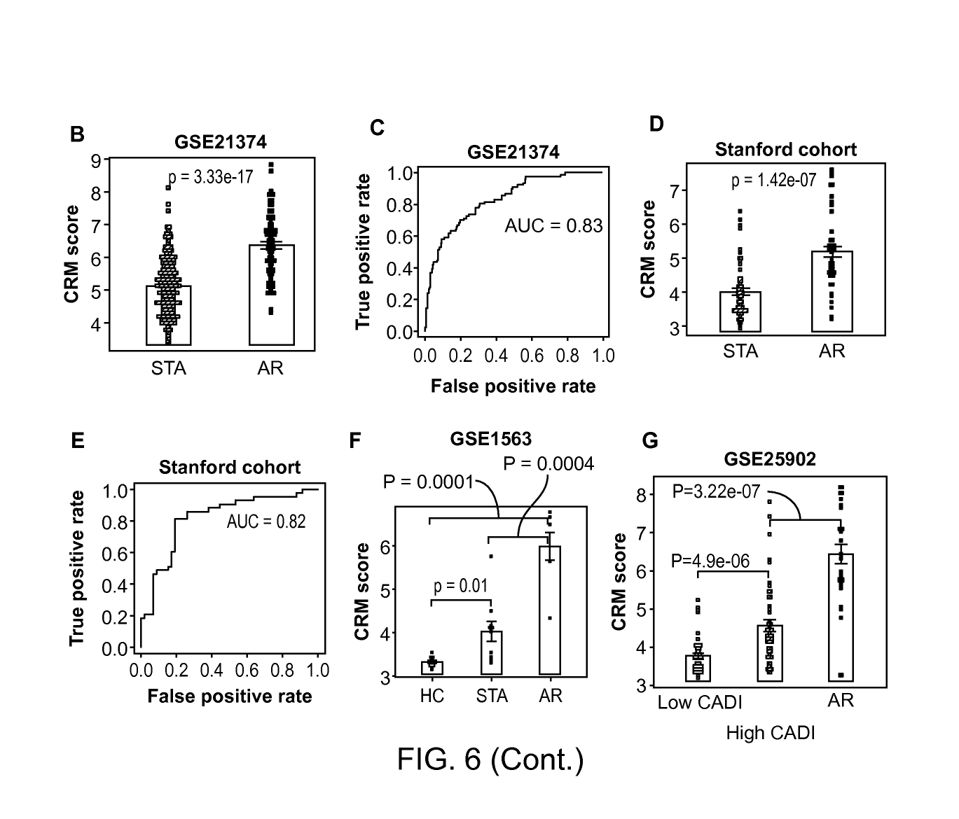

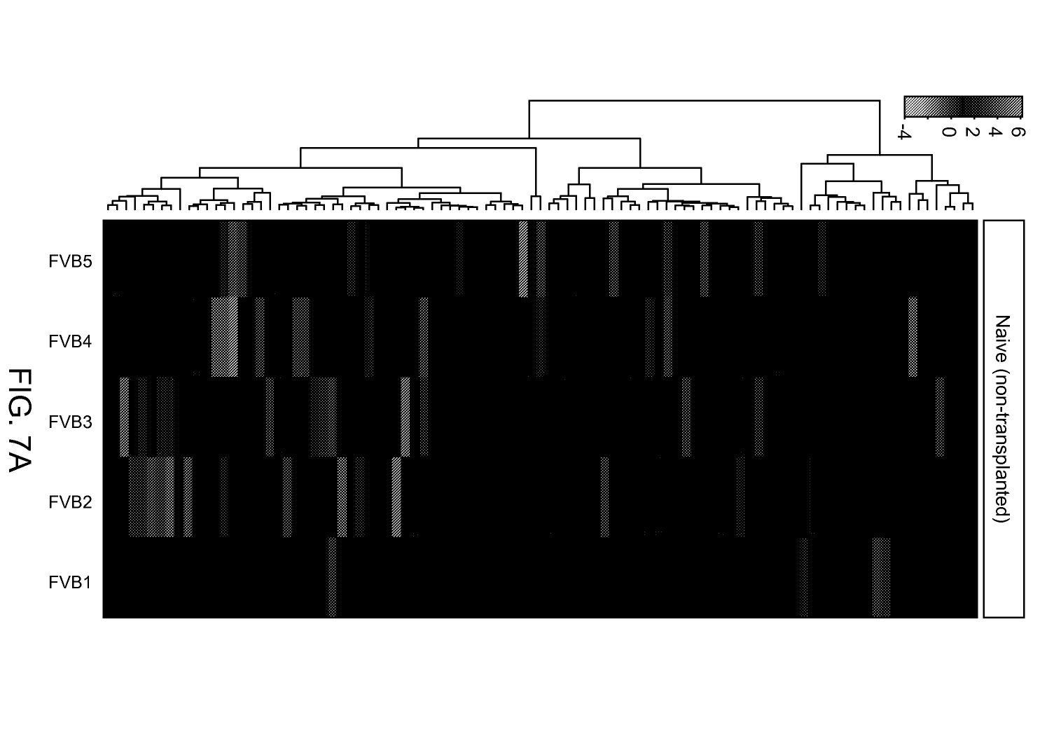

Overexpression of the 12 genes set were further validated in two independent cohorts consisting of 383 renal allograft biopsies. One of these data sets, GSE21374 (282 samples, AR=76, STA=206) (Einecke et al., 2010, J. Clin. Invest. 120:1862-1872) has previously been published. The second data set of 101 renal transplant biopsies (AR=43, STA=58) was generated for this study and, referred to as the “Stanford cohort” herein. All data sets contained biopsy-proven AR and STA renal allograft samples. Meta-analysis showed that all genes except CD7 were overexpressed in these cohorts (p<0.001, FDR<1%) in renal transplant biopsies during acute rejection (FIG. 6A). Consequently, CD7 was excluded from the list of 12 genes. The set of remaining 11 genes were defined as a common rejection module (CRM) that is an important transcriptional axis in acute rejection of transplanted solid organs.

D. Intragraft CRM Expression can Classify AR and STA Samples with High Accuracy.

The geometric mean of the CRM expression in each sample was defined as a CRM score. In each independent data set, the CRM score was significantly higher in AR group (p<5e-04; FIGS. 6B, 6D, and 6F). Each unit increment in the CRM score increased the odds of AR by 4.17 and 3.63 in GSE21374 and Stanford cohort, respectively. It was also able to distinguish AR and STA samples with high specificity and sensitivity in GSE21374 (AUC=0.83; FIG. 6C) and Stanford cohort (AUC=0.82; FIG. 6E).

E. Intragraft CRM Expression Correlates with Extent of Injury.

The CRM scores were further correlated with the extent of graft injury. In GSE1563, which also included healthy donor kidney biopsies, the CRM scores were the lowest for healthy donor kidney biopsies with very low variation (mean=3.31, s.d.=0.12), slightly higher for the STA samples (mean=4.0, s.d.=0.73), and the highest for AR (mean=5.98, s.d.=0.85) (FIG. 6F).

Furthermore, because the degree of progressive chronic histological damage is associated with long-term graft survival, we investigated relationship between the CRM scores and the chronic histological damage in renal allografts as defined by Chronic Allograft Damage Index (CADI) (Yilmaz et al., 2003, Journal of the American Society of Nephrology: JASN. 14:773-779). It was previously shown that there is a significant association between progressive histological damage in renal allografts and the intragraft expression of innate and adaptive immunity genes (Naesens et al., 2011, Kidney Int 80:1364-1376.). Gene expression data of 120 renal allograft biopsies were used from this study (GSE25902) and divided into the following three groups as defined in the previous study: low CADI (CADI<6), high CADI (CADI≧6) and AR. These three groups correspond to low, medium and high injury, respectively. It was determined that the CRM score increased with increased injury to an allograft (FIG. 6G), and were significantly different for each group (p<1E-05).

### F. FDA-Approved Drugs Targeting the CRM Genes Reduce Graft-Infiltrating Cells in a Murine Model of AR and Increase Graft Survival.

A literature review found that 6 out of the 11 genes are direct or indirect targets of FDA-approved drugs. Bortezomib is an FDA-approved drug that inhibits PSMB9. It can reverse antibody-mediated rejection and eliminate donor-specific anti-human leukocyte antigen antibodies (Walsh et al., 2010, Transplantation. 89:277-284). Mycophenolate mofetil, which is also FDA-approved and primarily targets IMP dehydrogenase 2 (IMPDH2), reduces expression of INPP5D by more than 2-fold (van Leuven et al., 2010, Atherosclerosis. 211:231-236). It is a potent immunosuppressive drug that reduces the risk of acute rejection (Knight et al., 2009, Transplantation. 87:785-794) and has a possible beneficial effect on chronic graft survival (Ojo et al., 2000, Transplantation. 69:2405-2409). BASP1 and CXCL9 are selectively targeted by doxycycline (Hartl et al., 2009, Proc Natl Acad Sci USA. 106:5604-5609) and sulindac (Sakaeda et al., 2006, Biochemical and Biophysical Research Communications. 350:339-344), respectively. LCK is one of the key targets of dasatinib (BMS-354825, Sprycel; Bristol-Meyers Squibb, New York, N.Y., USA), which is a drug that can inhibit T-cell activation (Lee et al., 2010, Leukemia. 24:896-900). Dasatinib is an ATP-competitor approved for Imatinib-resistant chronic myeloid leukemia. Atorvastatin (Lipitor) is an HMG-CoA reductase inhibitor that slows the production of cholesterol, which is used for treatment of hyperlipidemia. Out of nine chemokines and four endothelial cytokines investigated in plasma samples from patients with Crohn's disease, atorvastatin reduced CXCL10 plasma levels but did not affect the other chemokines and cytokines (Neurauter et al., 2003, Clinical Experimental Immunology. 131:264-267) (Grip and Janciauskiene, 2009, PLoS ONE. 4:e5263).

Both dasatinib and atorvastatin are FDA approved drugs for non-transplant conditions. Because atorvastatin and dasatinib have been FDA-approved for treating non-transplant conditions, they were further evaluated to determine whether they could be repositioned in organ transplantation. Both dasatinib and atorvastatin were tested in an established murine FVB-to-057BL/6 heterotopic cardiac transplant model to investigate the effect of peri-transplant drug administration on mitigating cell infiltration in the transplanted graft. The cardiac transplant model in mouse was chosen to also illustrate that the CRM is indeed common during AR in multiple organs. Cyclosporine was used as a positive control in this model. After each drug treatment, the grafts were evaluated by comparing against untreated AR using standard graft histology, a count of the infiltrating cell subsets in the graft, and by transcriptional analysis of the grafts Q-PCR.

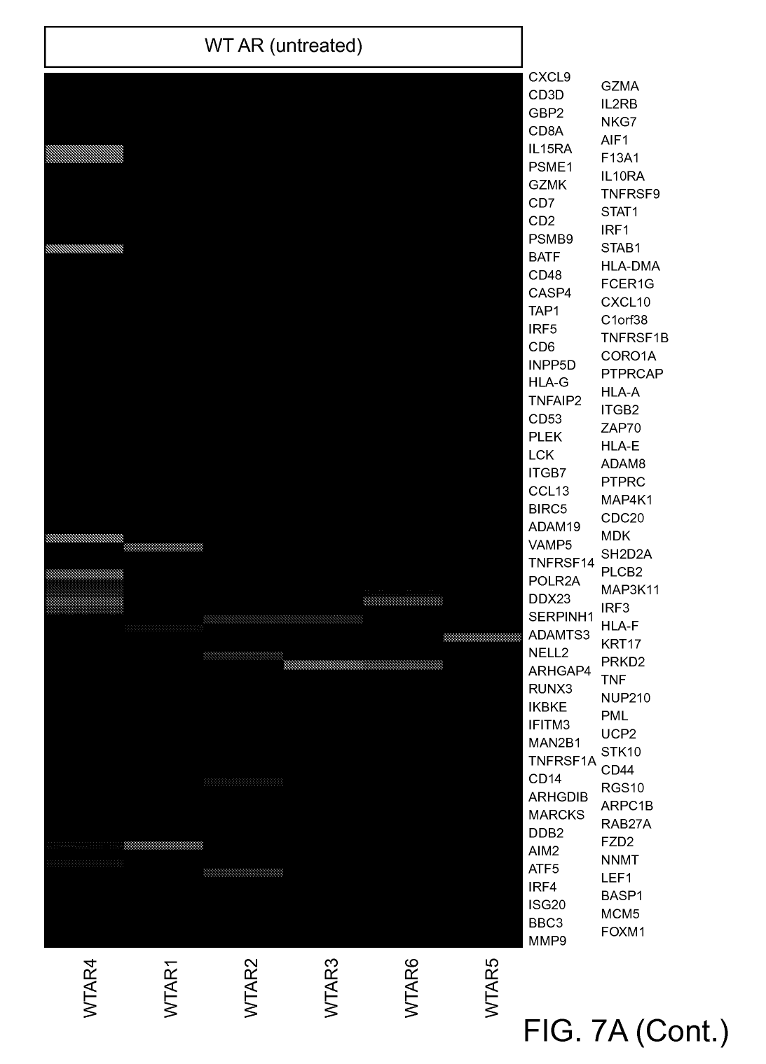

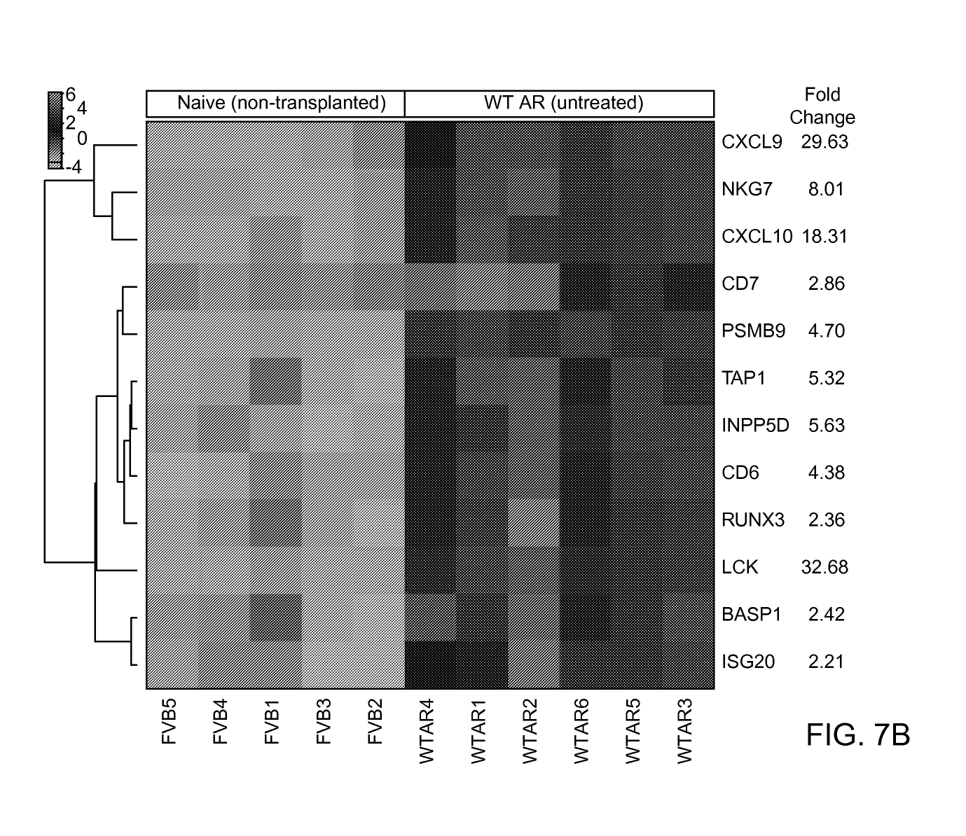

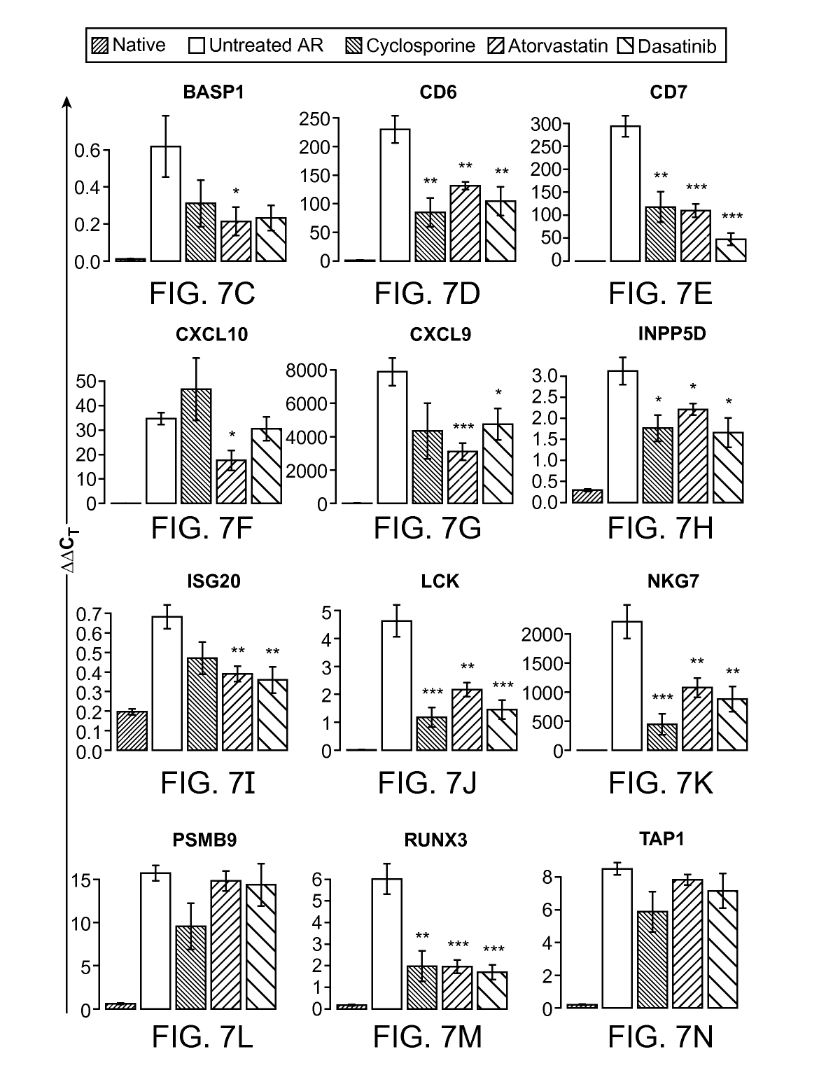

Gene expression profiling of non-transplanted hearts (FVB mice) and untreated, transplanted hearts showed that majority of the 102 cross-organ rejection genes were significantly overexpressed in untreated AR (FDR≦2%) (FIG. 7A), including all of the CRM genes (FDR≦0.1%; FIG. 7B). Pathway analysis of down-regulated genes in each treatment group against each untreated AR group using IPA showed that only cyclosporine affected the T cell-related pathways. Atorvastatin affected monocyte- and macrophage-related pathways, and dasatinib affected cell cycle-related pathways. Using Q-PCR, it was found that the majority of the CRM genes were down regulated in each of the treatment groups (FIG. 7C-N). Not all CRM genes were down regulated by any of the drugs used, which is expected because each drug acts through different mechanisms.

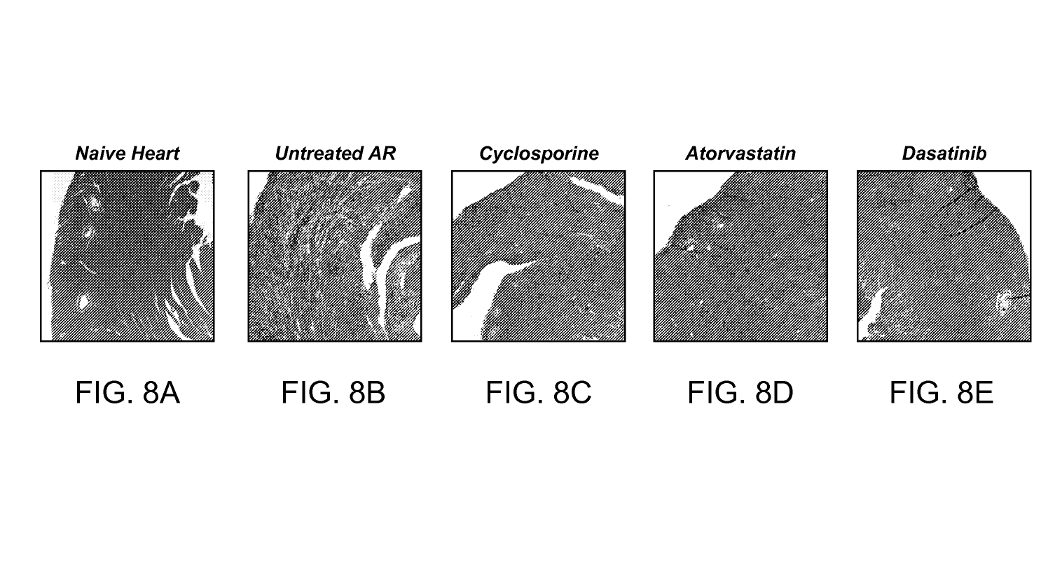

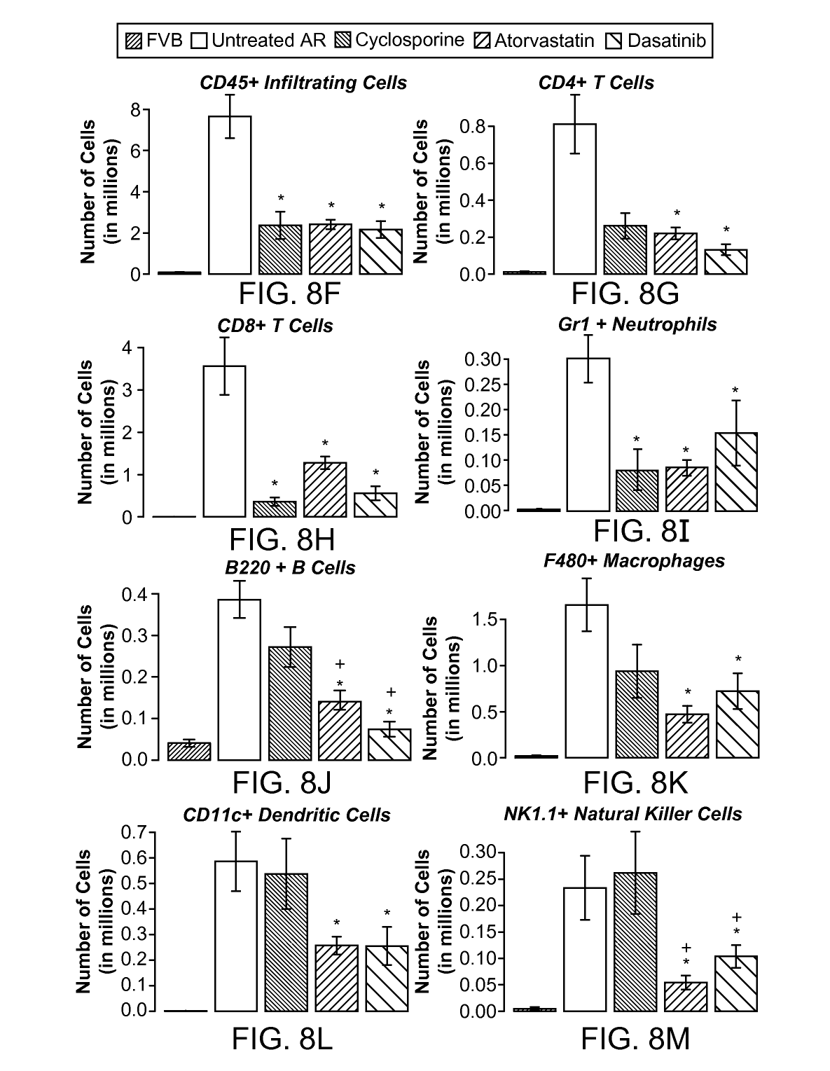

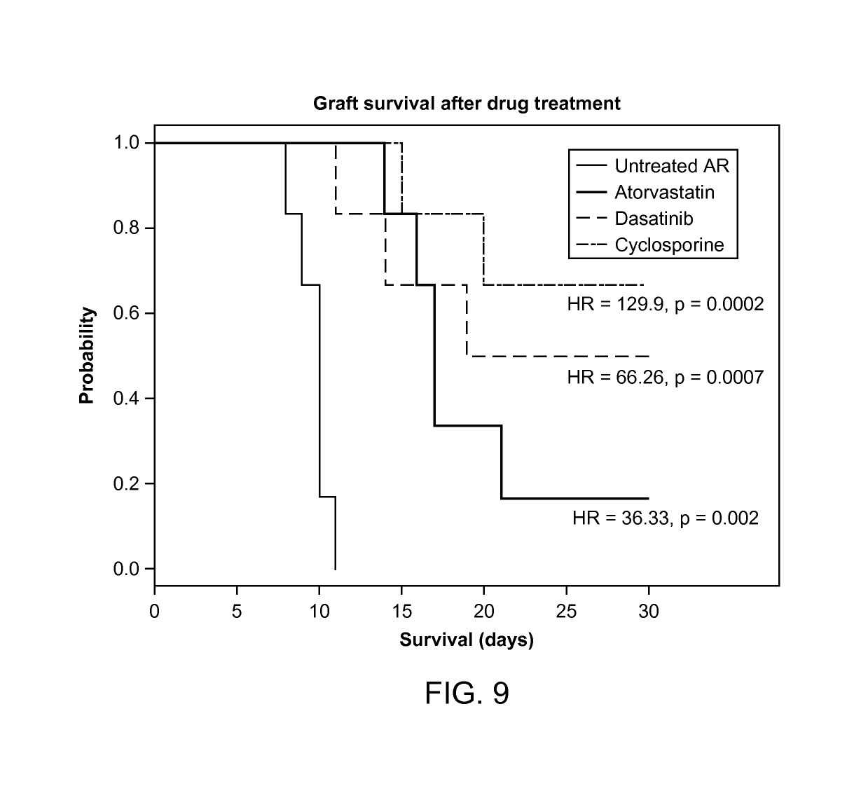

Furthermore, immunohistochemistry showed that there were fewer infiltrating cells in the atorvastatin and dasatinib treatment groups compared to untreated AR (p<0.005), and were equivalent to treatment with cyclosporine (p>0.05, i.e., statistically not significant) (FIG. 8A-F). For each treatment group, the number of total infiltrating CD45+ cells, CD4+ T cells, CD8+ T cells, B220+ B cells, CD11c+ dendritic cells, F4/80+ macrophages, Gr1+ neutrophils and NK1.1+ natural killer cells (FIG. 8F-M) were measured. Although all three drugs reduced CD4+ and CD8+ T cells compared to untreated AR, cyclosporine reduced the number of infiltrating CD8+ T cells significantly more than atorvastatin and dasatinib (FIG. 8G-H). However, atorvastatin and dasatinib reduced the number of infiltrating B220+ B cells compared to cyclosporine (FIG. 8J). Atorvastatin and dasatinib also reduced the number of infiltrating macrophages, dendritic cells and natural killer (NK) cells, while cyclosporine's effect was not statistically significant (FIG. 8K-M). Most notably, using Cox-proportional hazard analysis, when treated with atorvastatin or dasatinib compared to untreated AR, the hazard ratio for graft survival was 36.33 (p=0.002) and 66.26 (p=0.0007), respectively (FIG. 9). Median survival for the untreated AR group was 10 days, but was 17 days for atorvastatin and 24.5 days for dasatinib.

### G. Retrospective Analysis of Electronic Medical Records Shows Statin Treatment in Renal Transplant Patients Improves Graft Survival.

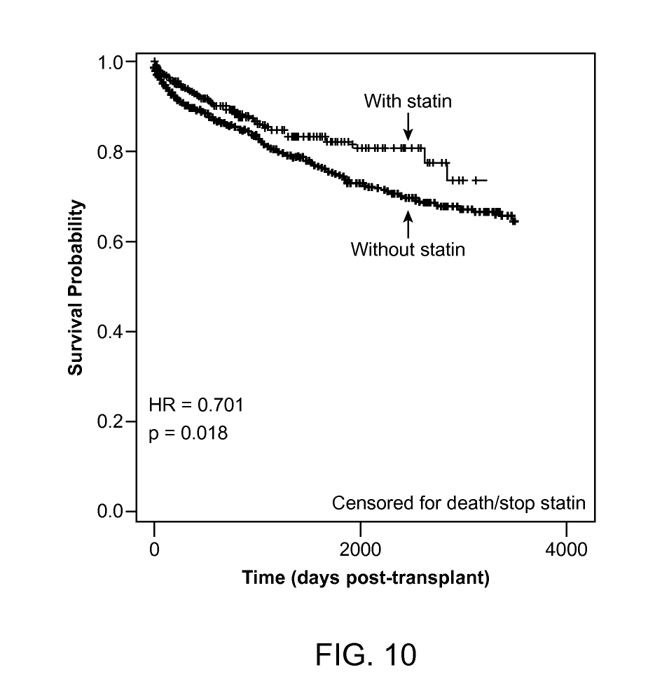

In order to validate the suggested benefits of statin use in a large clinical transplant population, electronic medical records were used from all 2,515 patients that received renal transplant between January 1989 and March 2012 at the University Hospitals Leuven (Leuven, Belgium). Out of the 2,515 patients, 1,566 received statin within the first 180 days after transplantation, with graft surviving at least 180 days. In Cox proportional hazards analysis, after censoring for when a patient stopped taking statin, graft failed or recipient death, statin use was associated with improved graft survival (HR=0.701 p=0.01) (FIG. 10). This effect was statistically significant after adjusting for donor and recipient age, repeat transplantation and calendar year.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of identifying a new therapeutic activity for a known therapeutic agent, the method comprising:
assessing samples from a plurality of subjects having a common condition for the presence of one or more biomarkers that are differentially expressed in the samples as compared to a control sample;
identifying a known therapeutic agent that modulates the activity of at least one differentially expressed biomarker whose presence is determined by the assessing, wherein the known therapeutic agent is not known to have therapeutic activity for the common condition; and
evaluating the therapeutic activity of the known therapeutic agent to treat the common condition.

2. The method according to claim 1, wherein the common condition is acute graft rejection.

3. The method according to claim 1, wherein the assessing comprises employing a meta-analysis protocol.

4. The method according to claim 1, wherein the known therapeutic agent is an agent that has been approved by a governmental agency for treatment in a disease condition that is different from the common condition.

5. A method for treating an acute graft rejection in a subject, the method comprising:
administering to the subject an effective amount of an active agent is selected from the group consisting of a BRC/ABL/Src tyrosine kinase inhibitor, a statin and combinations thereof.

6. The method of according to claim 5, wherein the inhibitor is a BRC/ABL/Src tyrosine kinase inhibitor.

7. The method according to claim 6, wherein the BRC/ABL/Src tyrosine kinase inhibitor is an amide substituted thiazole amine.

8. The method according to claim 7, wherein the amide substituted thiazole amine has the formula: 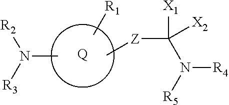
wherein
Q is thiazole;
Z is a single bond;
X₁ and X₂ together form ═O;
R₁ is hydrogen or alkyl;
R₂ is hydrogen or alkly;
R₃ is —Z₄—Z₆ wherein Z₄ is a single bond and wherein Z₆ is heteroaryl substituted with at least one group Z₃;
R₄ is hydrogen or alkly; and
R₅ is aryl which is unsubstitute or substitute with Z₁, Z₂ and one or more groups Z₃; and
Z₁, Z₂ and Z₃ are each independently
(1) hydrogen or Z₅, where Z₅ is (i) alkyl, alkenyl, alkynyl, cycoalkyl, cycloalkylalkyl, cycloalkenyl, cycloalkenylalkyl, aryl, aralkyl, alkylaryl, cycloalkylaryl, heterocyclo, or heterocycloalkyl; (ii) a group (i) which is itself substituted by one or more of the same or different groups (i); or (iii) a group (i) or (ii) which is substituted by one or more of the following groups (2) to (16) of the definition of Z₁, Z₂ and Z₃;
(2) —OH or —OZ₅;
(3) —SH or —SZ₅;
(4) —SH or —SZ₅;
(5) halo;
(6) cyano;
(7) nitro;
(8) oxo
(9) —O—C(O)—Z₅;
(10) any two of Z₁, Z₂ and Z₃ may together be alkylene or alkenylene completing a 3- to 8-membered saturated or unsaturated ring together with the atoms to which they are attached; or
(11) any two of Z₁, Z₂ and Z₃ may together be —O—(CH₂)ᵣ—O—, where r is 1 to 5, completing a 4- to 8-membered ring together with the atoms to which they are attached.

9. The method according to claim 8, wherein the amide substituted thiazole amine is dasatinib.

10. The method according to claim 5, wherein the active agent is a statin.

11. The method of according to claim 10, wherein the statin is a trans-6-[2-(3- or 4-carboxamido-substitute pyrrol-1-yl)alkyl]-4-hydroxypyran-2one or a derivative thereof.

12. The method according to claim 11, wherein the statin has the formula: 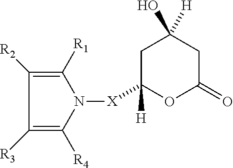
wherein
X is —CH₂—, —CH₂CH₂—, —CH₂CH₂CH₂—, or —CH₂CH(CH₃)—
R₁ is 1-naphtyl; 2-napthyl; cyclohexyl; norbornenyl; phenyl; phenyl substituted with fluorine; chlorine; bromine; hydroxyl; trifluoromethyl; alkyl of from one to four carbon atoms; alkoxy of from one to fourt carbon atoms; or alkanoyloxy of from two to eight carbon atoms;
either of R₂ or R₃ is —CONR₅R₆ where R₅ and R₆ are independently hydrogen; alkyl of form one to six carbon atoms; phenyl; phenyl substituted with fluorine, chlorine, bromine, cyano, trifluoromethyl, or carboalkoxy of from three to eight carbon atoms;
and the other of R₂ or R₃ is hydrogen; of from one to six carbon atoms; cyclopropyl; cyclobutyl; cyclopentyl; cyclohexyl; phenyl; or phenyl substituted with fluorine, chlorine, bromine, hydroxyl, trifluoromethyl, alkyl of from one to four carbon atoms, alkoxy of from one to four carbon atoms, or alkanoyloxy of from two to eight carbon atoms;
R₄ is alkyl of from one to six carbon atoms; cyclopropyl; cyclobutyl; cyclopentyl; cyclohexyl; or trifluoromethyl; or a hydroxyl acid or pharmaceutically acceptable salts thereof, corresponding to the opened ring of the compounds having the formula.

13. The method of according to claim 12, wherein the statin is atorvastatin.

14. The method according to claim 5, wherein the acute graft rejection is acute rejection of a solid organ graft.

15. The method according to claim 14, wherein the solid organ graft is selected from the group consisting of a heart, a kidney, a liver, a lung, and combinations thereof.

16. A pharmaceutical composition for the treatment of an allograft rejection comprising an effective amount of at least one of a BRC/ABL/Src tyrosine kinase inhibitor and a statin in combination with a known graft rejection active agent.

17. The pharmaceutical composition according to claim 16, wherein the BRC/ABL/Src tyrosine kinase inhibitor is dastinib.

18. The pharmaceutical composition according to claim 16, wherein the statin is atorvastatin.

19. The pharmaceutical composition according to claim 16, wherein the known graft rejection active agent is cyclosporin.

20. (canceled)
